SEQUENCE LISTING (26 sequences)

SEQ ID NO: 1 - protein, 7 aa, synthetic construct
EMVLLER

SEQ ID NO: 2 - protein, 12 aa, synthetic construct
SGQYLQNSTSSR

SEQ ID NO: 3 - DNA, 18 bp, synthetic construct
  source 1..18: mol_type = other DNA
ccaaatgtactacaacta

SEQ ID NO: 4 - DNA, 60 bp, synthetic construct
  source 1..60: mol_type = other DNA
ctgctggagcggctgcggcacccaaatgtactacaactatatggctactgctaccaggac

SEQ ID NO: 5 - DNA, 59 bp, synthetic construct
  source 1..59: mol_type = other DNA
tcctggtagcagtagccatatagttgtagtacatttgggtgccgcagccgctccagcag

SEQ ID NO: 6 - protein, 16 aa, synthetic construct
CKALKAVDFSGHDLGS

SEQ ID NO: 7 - DNA, 22 bp, synthetic construct
  source 1..22: mol_type = other DNA
gaagcggaacctctataatgcc

SEQ ID NO: 8 - DNA, 22 bp, synthetic construct
  source 1..22: mol_type = other DNA
ggatacactggtactcggtact

SEQ ID NO: 9 - DNA, 22 bp, synthetic construct
  source 1..22: mol_type = other DNA
gaggagtacgtctacttcagcc

SEQ ID NO: 10 - DNA, 20 bp, synthetic construct
  source 1..20: mol_type = other DNA
cggtgggaaaagtcactgtc

SEQ ID NO: 11 - DNA, 20 bp, synthetic construct
  source 1..20: mol_type = other DNA
tggactgcctcccgattcct

SEQ ID NO: 12 - DNA, 20 bp, synthetic construct
  source 1..20: mol_type = other DNA
ggctgatgcagaggcacaga

SEQ ID NO: 13 - DNA, 19 bp, synthetic construct
  source 1..19: mol_type = other DNA
atggggaaggtgaaggtcg

SEQ ID NO: 14 - DNA, 22 bp, synthetic construct
  source 1..22: mol_type = other DNA
ggggtcattgatggcaacaata

SEQ ID NO: 15 - DNA, 20 bp, synthetic construct
  source 1..20: mol_type = other DNA
cggtcaccctgcttgacttc

SEQ ID NO: 16 - DNA, 22 bp, synthetic construct
  source 1..22: mol_type = other DNA
ggcattgtagaggttccgtttc

SEQ ID NO: 17 - DNA, 20 bp, synthetic construct
  source 1..20: mol_type = other DNA
gccctcagcttcctggacat

SEQ ID NO: 18 - DNA, 20 bp, synthetic construct
  source 1..20: mol_type = other DNA
acgtcaatagctaccaccgt

SEQ ID NO: 19 - DNA, 20 bp, synthetic construct
  source 1..20: mol_type = other DNA
gccagcttctctccctcctt

SEQ ID NO: 20 - DNA, 20 bp, synthetic construct
  source 1..20: mol_type = other DNA
aggtgggaagccagtgagtt

SEQ ID NO: 21 - DNA, 21 bp, synthetic construct
  source 1..21: mol_type = other DNA
tcagtcaacgggggacataaa

SEQ ID NO: 22 - DNA, 21 bp, synthetic construct
  source 1..21: mol_type = other DNA
ggggctgtactgcttaaccag

SEQ ID NO: 23 - DNA, 28 bp, synthetic construct
  source 1..28: mol_type = other DNA
ctacagataatctcctttaatgggtggg

SEQ ID NO: 24 - DNA, 26 bp, synthetic construct
  source 1..26: mol_type = other DNA
cttctacgcagagtacttcagaaacc

SEQ ID NO: 25 - DNA, 20 bp, synthetic construct
  source 1..20: mol_type = other DNA
gactccttggtcttacggag

SEQ ID NO: 26 - DNA, 20 bp, synthetic construct
  source 1..20: mol_type = other DNA
ctccaccagctttagccagc

(19) United States
(12) Patent Application Publication
(10) Pub. No.: US 2025/0289906 A1
(43) Pub. Date: Sep. 18, 2025

(54) MODULATION OF EXTRACELLULAR KINASE ACTIVITY AND FUNCTION FOR TREATMENT OF PAIN AND NEURODEGENERATIVE/NEUROIMMUNE DISEASE

(71) Applicants: BOARD OF REGENTS, THE UNIVERSITY OF TEXAS SYSTEM, Austin, TX (US); THOMAS JEFFERSON UNIVERSITY, Philadelphia, PA (US)

(72) Inventors: Theodore J. PRICE, Dallas, TX (US); Matthew B. DALVA, Philadelphia, PA (US)

(73) Assignees: BOARD OF REGENTS, THE UNIVERSITY OF TEXAS SYSTEM, Austin, TX (US); THOMAS JEFFERSON UNIVERSITY, Philadelphia, PA (US)

(21) Appl. No.: 19/054,404

(22) Filed: Feb. 14, 2025

Related U.S. Application Data
(60) Provisional application No. 63/554,730, filed on Feb. 16, 2024.

(51) Int. Cl. C07K 16/40; A61K 39/00; A61P 25/04; C07K 16/28; C12N 15/86

(52) U.S. Cl. CPC C07K 16/40; A61P 25/04; C07K 16/2866; C12N 15/86; C12Y 207/10002; A61K 2039/505; C12N 2740/15043

(57) ABSTRACT

The present disclosure is directed to modulation of extracellular kinase activity, such as inhibiting VLK activation and function, for treatment of pain and neurodegenerative/neuroimmune disease.

### PRIORITY CLAIM

This application claims benefit of priority to U.S. Provisional Application Ser. No. 63/554,730, filed Feb. 16, 2024, the entire contents of which are hereby incorporated by reference.

### STATEMENT REGARDING FEDERALLY SPONSORED RESEARCH AND DEVELOPMENT

This invention was made with government support under RO1 NS115441 and RO1 NS111976 awarded by the National Institutes of Health. The government has certain rights in the invention.

### REFERENCE TO A SEQUENCE LISTING

This application contains a Sequence Listing XML, which has been submitted electronically and is hereby incorporated by reference in its entirety. Said Sequence Listing XML, created on May 15, 2025, is named UTSFP0165US.xml and is 24,048 bytes in size.

### BACKGROUND

### 1. Field of the Disclosure

The present disclosure relates generally to the fields of medicine, neurobiology and pain management. More particularly, the disclosure relates to the blocking of Vertebrate Lonesome Kinase (VLK) kinase function and activation in subjects suffering from pain and neurological conditions.

### 2. Background

Mass spectrometry analysis of extracellular protein domains has identified thousands of phosphorylation events on serine, threonine, and tyrosine residues. These phosphorylations, believed to be mediated by two families of kinases, have an underexplored functional significance in the nervous system. Notably, mutations in FAM20C, a serine/threonine-directed kinase, result in Raine syndrome, highlighting the importance of these kinases. Similarly, the tyrosine-directed Vertebrate Lonesome Kinase (VLK) is an essential gene in mice, suggesting its crucial role in biological processes. However, a better understanding of the role phosphorylation plays in these events, and in particular that of VLK, is needed.

### SUMMARY

Thus, in accordance with the disclosure, there is provided a method of targeting Vertebrate Lonesome Kinase (VLK) activity in a subject for the treatment of acute pain, such as that induced by disease, injury and/or surgery, or for the treatment of a neurological disorder, comprising delivering to said subject a therapy that blocks VLK kinase activity or activation or that interferes with VLK interaction with EphB receptors, such as by delivery to thalamus, anterior cingulate cortex, dorsal root ganglion and/or spinal cord of the subject. Also provided is a method of treating a neurological disorder linked to the EphB-NMDAR interaction selectively induced by VLK, such as Alzheimer's, Schizophrenia, NMDAR encephalitis, Autism Spectrum Disorder, or stroke, comprising delivering to said subject a therapy that blocks VLK kinase activity or activation or that interferes with VLK interaction with EphB receptors, such as by delivery to brain cortex or delivery to the limbic system of the subject. The therapy may be a drug blocking activating VLK kinase activity, an antibody or fragment thereof blocking VLK activity, a drug or antibody that interferes with VLK interaction with EphB receptors, or a drug or antibody that interferes with EphB-NMDAR interaction. The pain may be acute pain, cancer pain, neuropathic pain, injury-induced pain, or NMDAR dependent pain. The step of delivering may be repeated, such as repeated 1, 2, 3, 4, 5, 6, 7, 8, 9, 10, 15, 20, 25, 50, 75, or 100 times or is given chronically, such as daily or weekly over 1, 2, 3, 4, 5, 6, 7, 8, 9, 10, 11, 12 or 24 months.

Also provided is a method of treating a chronic disease associated with EphrinB upregulation, such as rheumatoid arthritis, inflammatory bowel disease, stroke and neurological disorders in a subject, comprising targeting VLK activity in said subject with a drug blocking activating VLK kinase activity, an antibody or fragment thereof blocking VLK activity, a drug or antibody that interferes with VLK interaction with EphB receptors, or a drug or antibody that interferes with EphB-NMDAR interaction. The disease may be characterized by EphrinB driving VLK release from dorsal root ganglion peripheral neurons and/or from central nervous system neurons. The step of targeting may be repeated, such as repeated 1, 2, 3, 4, 5, 6, 7, 8, 9, 10, 15, 20, 25, 50, 75, or 100 times or is given chronically, such as daily or weekly over 1, 2, 3, 4, 5, 6, 7, 8, 9, 10, 11, 12 or 24 months.

Further provided is a method for treating rheumatoid arthritis, inflammatory bowel disease, or a neurological disorder such as Alzheimer's, Schizophrenia, NMDAR encephalitis, autism spectrum disorder, or stroke in a subject comprising inhibiting JAK/STAT signaling to disrupt increased VLK. Inhibiting JAK/STAT signaling may comprise administration of a drug blocking activating VLK kinase activity, an antibody or fragment thereof blocking VLK activity, a drug or antibody that interferes with VLK interaction with EphB receptors, or a drug or antibody that interferes with EphB-NMDAR interaction. The administration may be repeated, such as repeated 1, 2, 3, 4, 5, 6, 7, 8, 9, 10, 15, 20, 25, 50, 75, or 100 times or is given chronically, such as daily or weekly over 1, 2, 3, 4, 5, 6, 7, 8, 9, 10, 11, 12 or 24 months.

In yet another embodiment, there is provided a method of treating pain in a subject in need thereof comprising using targeted CRISPR gene excision, viral vector mediated gene knockdown, antisense oligonucleotide or RNA interference to knockout or knockdown VLK expression in sensory neurons of said subject.

An even further embodiment provides a method of determining whether a patient will respond to a VLK-targeting therapeutic comprising (a) administering an NMDA receptor antagonist to said subject; and (b) assessing the presence or absence of an acute analgesic response to an NMDA receptor antagonist, wherein an acute analgesic response indicates that said subject will respond to a VLK-targeting therapeutic. The method may further comprise (c) treating said subject with a VLK-targeting therapeutic when an acute analgesic response occurs. The NDMA receptor antagonist may be ketamine or memantine. The method may further comprise, in the absence of an acute analgesic response, repeating step (a) at a higher dose of said NMDA receptor antagonist. The subject may be a mammal, such as a human.

Yet a further embodiment provides a method of targeting an agent to an EphB2-NMDAR complex in a subject comprising (a) providing an agent linked to Vertebrate Lonesome Kinase (VLK) or a fragment thereof that interacts with EphB2 receptors but lacks VLK activity, and (b) administering said VLK-linked agent to a subject. The agent may be an inhibitor or activator of VLK.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

The patent or application file contains at least one drawing executed in color. Copies of this patent or patent application publication with color drawing(s) will be provided by the Office upon request and payment of the necessary fee.

FIGS. 1A-K: VLK is sufficient to induce EphB2-NMDAR interaction. FIG. 1: VLK is sufficient to induce the EphB2-NMDAR interaction. (FIG. 1A) Immunoblotting for co-immunoprecipitated GluN1 with EphB2 in stable VLK knockdown HEK293T cells. Control (C) cells were transfected with GluN1, GluN2B, and EphB2. 293T Cells in other experimental conditions were transfected with each indicated VLK related kinase VLK, Fam69A, Fam69B, Fam69C, DIA1, or DIA1R along with EphB2, GluN1 and GluN2B. Levels of EphB2 immunoprecipitated from cell lysates are shown by immunoblotting for EphB2 (IP Controls, middle). Extracellular kinases expression levels in cell lysates are shown by immunoblotting for FLAG after FLAG immunoprecipitation (FLAG, IP Controls, bottom). (FIG. 1B) Immunoblotting to detect the expression of secreted kinases in cell-conditioned ACSF (45 min.) from HEK293T cells transfected with kinases described above (ACSF-IP, top). Cell-conditioned ACSF and cell lysates (Cyto, bottom) were Immunoprecipitated with a monoclonal anti FLAG antibody and immunoblotted with a polyclonal anti FLAG antibody. (FIG. 1C) Untransfected cultured cortical neurons (DIV7-8) treated with clustered EphrinB2 (+eB2) or control (C) reagents in ACSF for 45 min. Phospho-Tyrosine specific antibody pY99 was used to immunoprecipitate conditioned ACSF and immunoblotted with a custom-generated VLK antibody (ACSF-IP, top). Conditioned ACSF (ACSF, middle) and cell lysates (Cyto, bottom) were probed with beta-actin as a control to show that the conditioned media was free of any intracellular protein. (FIG. 1D) iPSC-derived sensory Dorsal Root Ganglion neurons (DRGx) cultured for four weeks and subject to identical treatment and experimental conditions for immunoprecipitation and immunoblotting as in FIG. 1C above. (FIG. 1E) Representative images of PLA of EphB2-NMDAR after treatment with soluble recombinant kinases. HEK293T cells were transfected with GluN1, GluN2B, EphB2, and EGFP. Cells were treated with active recombinant VLK, DIA1, or Fam69C proteins (as indicated) for 45 minutes before fixation. The upper panels show PLA signal alone (magenta). The lower panels are the merged images with EGFP in green. Scale bar is 10 μm. (FIG. 1F) Quantification of the PLA puncta number. PLA puncta numbers are quantified by counting the puncta per 100 μm² in EGFP⁺ cells and normalized to the Control. (****p=<0.0001,*p=0.038 One way ANOVA followed by Tukey's; Control n=70 fields from 7 repeats; VLK n=70 fields from 7 repeats; DIA1 n=40 fields from 4 repeats; Fam69C n=40 fields from 4 repeats -). (FIG. 1G) Untransfected cultured cortical neurons (DIV7-8) treated with control reagents (C), clustered EphrinB2 (+eB2), recombinant VLK (+VLK) or recombinant kinase dead VLK (+VLK-KD) for 45 min. EphB2 was immunoprecipitated from the cell lysates, and co-immunoprecipitated GluN1 was immunoblotted (Co-IP, top). A fraction of the same sample was immunoblotted for EphB2 (EphB2, IP). Neuronal lysates were probed as lysate controls indicated for EphB2, GluN1, and the intracellular phosphorylation of EphB2 was probed using EphB2 pY662. (FIG. 1H) Representative images of PLA of EphB2-NMDAR in CD1 mouse brain synaptosomes after treatment with recombinant VLK kinase with or without ATP or with ATP along with VLK with PAP phosphatase (as indicated). The upper panels show the PLA signal alone (magenta). The lower panels are merged images of synaptosomes immunostained for a presynaptic marker vGlut1 (green). The no antibody control condition was performed without primary antibody for EphB2 and GluN1. Scale bars is 5 μm. (FIG. 1I) Quantification of the percentage of vGlut1⁺ synaptosomes that colocalize with PLA puncta. (n=40 fields from 4 different mice, One Way ANOVA followed by Tukey's test ****p<0.0001. (FIG. 1J) PLA performed from synaptosomes derived from Human Spinal cord. Scale bars is 5 μm. (FIG. 1K) Quantification of the percentage of vGlut1⁺ synaptosomes that colocalize with PLA puncta. (n=30, fields from 3 different human samples, One Way ANOVA followed by Tukey's test, *p=0.04,0.04, **p=0.006, 0.0067).

FIGS. 2A-I: VLK is necessary for the EphB2-NMDAR interaction. (FIG. 2A) In vitro experimental approach to test EphB2 extracellular domain (EphB2-ectodomain) phosphorylation (yellow circle P) by active recombinant VLK (VLK, purple) or kinase-dead VLK (VLK-KD, orange). (FIG. 2B) In vitro kinase assay to test the tyrosine phosphorylation of EphB2 extracellular domain (EphB2-ec) by kinase active recombinant VLK (rVLK) or kinase-dead recombinant VLK (rVLK-KD) in ACSF, and MnCl₂. The extracellular domain of EphB2 fused to the Fc fragment of immunoglobulin G (200 ng) was incubated at 37° C. for 30 min. with rVLK or rVLK-KD (100 ng), either with or without ATP in MgAc (1 mM) as indicated. EphB2-Fc was precipitated with Protein G agarose beads and probed with a phosphor-tyrosine antibody pY99 and EphB2. Blots were probed as shown. (FIG. 2C, top row) In vitro kinase assay to test the tyrosine phosphorylation of EphB2 extracellular domain (EphB2-ec) by kinase active recombinant Fam69C (rFam69C) and Dia1 (rDia1). The experiment was set up as described in FIG. 2B above. rVLK has been included as a positive control for comparison. Blots were probed as shown. (FIG. 2C, second row, FIG. 2D, and FIG. 2E) Model illustrating VLK (purple) dependent extracellular phosphorylation (yellow circle P) at wildtype EphB2 tyrosine residue Y504 introduces a negative charge on EphB2 that is necessary for its interaction with the positively charged NMDAR N-terminal domain (FIG. 2C, second row). VLK cannot induce EphB-NMDAR interaction in the presence of a neutrally charged phosphor-null mutant Y504F (grey circle FIG. 2F) in ®, and the interaction is constitutive in the presence of the negatively charged phosphor-mimetic mutant Y504E in the absence of VLK (FIG. 2G). HEK 293T cells with stable VLK knockdown were transfected with GluN1, GluN2B, and either VLK or kinase-dead VLK (VLK-KD), and with wild type EphB2 in (FIG. 2C second row), EphB2 Y504F in ® or EphB2 Y504E (FIG. 2E). The cell lysates were immunoprecipitated for EphB2 and immunoblotted to detect co-immunoprecipitated GluN1. A fraction of the same EphB2 immunoprecipitated sample was immunoblotted for EphB2 (EphB2, IP), and lysate controls were probed as indicated. VLK blot is generated as described in FIG. 1B. (FIG. 2F) A schematic illustrating the conditional knockout of the PKDCC gene that encodes for the VLK protein. PKDCC floxed (fl) mice harboring loxP sites flanking the Exon 1 of the PKDCC gene were bred with CamKII-Cre to generate an excitatory cell-specific knockout PKDCC^{fl/fl} CamKII^{Cre/+} mice. (FIG. 2G) Real-Time (RT) PCR based quantification of relative mRNA levels of PKDCC (VLK), DIPK1C (Fam69C), and DIPK2B (Dia1R) from the cortices of PKDCC^{fl/fl} CamKII^{Cre/+} mice (−/−, grey bars) compared to control mice cortices (+/+, black bars). (***p<0.0001, unpaired t-test; n=3). (FIG. 2H) Immunoblotting for co-immunoprecipitated GluN1 with EphB2 from cortical lysates of PKDCC^{fl/fl}CamKII^{Cre/+}(KO, top), compared to wild type (WT, top) littermate controls. A fraction of the same EphB2 immunoprecipitated sample was immunoblotted for EphB2 (EphB2, IP). pY99 immunoprecipitation from cortical lysate was immunoblotted for co-immunoprecipitated VLK (VLK, IP). Cortical lysates were probed as indicated for EphB2 and GluN1 (Lysate).

FIGS. 3A-Q: VLK is a synaptic protein and functions by being released presynaptically by DRG neurons. (FIG. 3A) Western blots of PSD purification fractions prepared from WT CD1 mouse brain show VLK is enriched in crude vesicle fraction (S3). Gels were loaded with non-synaptic (S1), crude synaptosomal (P2), crude synaptic vesicle (S3), synaptic plasma membrane (SPM), and post-synaptic density (PSD) fractions. PSD fractions (from left to right) are least insoluble first triton extraction (1T), second triton extraction (2T), and most insoluble sarcosyl extraction(S). Blots were probed with VLK, Syp1, GluN1, GluN2B, and PSD-95 (as indicated). (FIG. 3B) The First and Last frame of the same axon segment from cultured rat cortical neurons (DIV21-23) transfected with VLK-mCherry (VLK, magenta), Synaptophysin1-EGFP (Syp1, green). mTurquoise was transfected as a cell fill (omitted for clarity). Kymograph showing live cell imaging of VLK and Syp1. The same axon segment was fixed after live imaging and stained for PSD-95 (Posthoc, cyan). Scale bar is 5 μm. Yellow arrowhead shows an example of colocalized VLK and Syp1 that remain stationary during live imaging and colocalize with PSD95. Magenta arrowhead shows an example of colocalized VLK and Syp1 that remain stationary during live imaging but do not colocalize with PSD95. Cyan arrowhead shows an example of Syp1 that remains stationary and colocalized with PSD95. (FIG. 3C) The individual channels for VLK, Syp1, PSD95 and Merged are shown for the same axon segment as FIG. 3B above. Scale bar is 5 μm (FIGS. 3D-E) Graph depicting the puncta density of stable and moving VLK (magenta), Syp1 (green) and VLK+Syp1 (gray). (FIG. 3F) Graph depicting the puncta density of colocalized puncta after posthoc staining with PSD95 (95) VLK and Syp1 that remained stationary during live imaging and were maintained after fixation were quantified. (FIG. 3G) Schematic illustrating the experiment to test if ephrin B2 (eB2) stimulated VLK secretion from neurons can be blocked by botulinum toxins, A or B (BoNT A/B). (FIG. 3H) Cultured cortical neurons were transduced with lentivirus expressing VLK-mCherry at DIV3 and assayed at DIV7-8. Neurons were pretreated with BoNT A or B for 60 min followed by treatment with clustered EphrinB2 (+eB2) or control (C) reagents with or without toxins in ACSF for 45-60 min. Cell-conditioned ACSF were immunoprecipitated with RFP Trap agarose and immunoblotted for mCherry (top blot, ACSF IP). Cell-conditioned ACSF were probed for beta-actin to show no intracellular protein leaked into the ACSF (Actin, Sup). Cell lysates were probed for mCherry, EphB2 and EphB2 pY662 (as indicated). (FIG. 3I) RNAscope in situ hybridization for PKDCC (Red) in mouse lumbar DRG, in Scn10a nociceptors (cyan) and Merged overlay with Nf200 mechanoreceptor cells (blue). Scale bar is 50 μm. (FIG. 3J) Mouse Dorsal Root Ganglion (DRG) have both nociceptor and mechanoreceptor cells marked by Scn10a and Nf200 respectively. (FIG. 3K) Quantification of High PKDCC expressing neurons (n=8, Nf200 Mean=23.83, SEM±4.490, Scn10a Mean=10.95, SEM±2.740). (FIG. 3L) RNAscope in situ hybridization for PKDCC (Red) in human lumbar DRG, Scn10a (green), Lipofuscin (white), DAPI (blue) Scale bar is 50 μm. Merged overlay, scale bar is 50 μm. Inset from overlay showing PKDCC expression in neurons (white arrows) Scale bar is 50 μm?*. (FIG. 3M) Quantification of PKDCC expressing neurons (n=8, PKDCC Mean=21.41, SEM±2.156; SCN10A Mean=75.98, SEM±1.509). (FIG. 3N) Quantified percentage of PKDCC expression in nociceptor and non-nociceptor populations. (n=8, Non-nociceptor Mean=35.82, SEM±2.706; Nociceptor Mean=16.95, SEM±2.443). (FIG. 3O) A model of the DRG from PKDCC conditional knockout mice (cKO). PKDCC-flox mice were crossed to Pirt-Cre line to generate PKDCC sensory-neuron specific cKO mice (FIG. 3P) RNAscope in situ hybridization for PKDCC to confirm PKDCC deletion from sensory neurons in PKDCC cKO mice. Scale bar is 50 μm (FIG. 3Q) Model of WT and cKO mice (as described above) detailing the experiment to test the effect of PKDCC deletion in the presynaptic DRG sensory neurons on the EphB2-NMDAR interaction in the dorsal horn (DH) postsynaptic neurons. ® Immunoblot showing levels of GluN1 co-immunoprecipitated with EphB2 in the DH of cKO mice compared to wildtype littermate controls (WT). A fraction of the same sample was immunoblotted for EphB2 (EphB2, IP). DH lysates were probed as indicated for EphB2 and GluN1.

FIGS. 4A-W: Role of VLK in pain models. (FIG. 4A) Schematic of intrathecal drug administration and measurement of pain-like behaviors. von-Frey testing is used as a measure of mechanical sensitivity and grimace scores depict levels of affective pain. (FIG. 4B, FIG. 4C) Three different secreted kinases and their denatured controls were administered to mice (concentrations indicated): VLK, DIA-1 and FAM69C. Mice receiving VLK displayed a sharp drop in mechanical thresholds and increased grimacing. (n=4, Two-way repeated measures ANOVA, F (5, 162)=82.33, **p=0.001, **** P<0.0001; F (5, 162)=9.759, ***p<0.0001. post hoc Dunnett's test). (FIG. 4D, FIG. 4E) Administration of a kinase-dead mutant of VLK (VLK-KD) abolishes pain-like behaviors induced by VLK. (n=5, Repeated measures ANOVA, F (2, 11)=26.19, ****p<0.0001; Repeated measures ANOVA, F (2, 11)=11.07, **p0.0023. post hoc Dunnett's test). (FIG. 4F) Schematic of administration of the extracellular phosphatase PAP followed by VLK and measurement of pain-like behaviors. (FIG. 4G, FIG. 4H) Mice administered PAP followed by VLK showed significantly reduced mechanical sensitivity (n=8, Two way repeated measures ANOVA, F (5, 70)=18.96, ***p=0.0003 Posthoc: Bonferroni) whereas PAP did not affect VLK-induced grimacing compared to denatured PAP control (n=8, Two way repeated measures ANOVA, Posthoc: Bonferroni). (FIG. 4I, FIG. 4J) Co-administration of VLK with AP5 also blocked the VLK-induced pain phenotype. (n=8, Repeated Measures ANOVA F (1, 18)=18.85, P=0.0004, F (1, 18)=19.93, P=0.0003. post hoc Bonferroni's test). (FIG. 4K) The lumbar spinal cord dorsal horn from mice administered intrathecal heat Denatured VLK, VLK, or VLK+AP5 was immunohistochemically stained for NeuN and cFos markers. (FIG. 4L) Quantification of cFos⁺ cells showed VLK induced a significant increase in neuronal cFos expression which was blocked by AP5 antagonism of NMDARs or with the use of heat Denatured VLK (D-VLK). (One-way ANOVA with Bonferroni multiple comparisons ****p<. 0001, ***p<. 001). (FIG. 4M) Illustration depicting intrathecal injection of recombinant VLK in EphB2 TacR1 Cre+/+ mice (EphB2 cKO). (FIGS. 4N-O) Measurement of pain-like behaviors using Von Frey(N) (n=7, Two way repeated measures ANOVA, F (5, 65)=2.641, **p=0.003 and Grimace(O) (n=7, p=NS) (FIG. 4P) Model depicting PKDCC^{fl/fl} Pirt^{Cre/+} mice (cKO) and PKDCC^{fl/fl} littermate controls (Control) were subjected to behavioral testing for normal sensorimotor development. (FIGS. 4Q-S) Paw withdrawal latencies for were measured on a Hot Plate in (FIG. 4Q) and Hargreaves assay in (FIG. 4R). Rotarod testing was performed in(S). No significant differences were observed between genotypes in the above tests. (FIG. 4T) A model of the PKDCC conditional knockout mice (cKO). VLK-flox mice were crossed to Pirt-Cre line to generate PKDCC (VLK⁺) sensory-neuron specific cKO mice. cKO and wild-type littermates were subjected to Hind-paw incision surgeries. (FIG. 4U, FIG. 4V) Behavioral data showing effects of sensory neuron-specific VLK knockout on postsurgical mechanical hypersensitivity and affective pain. (n=9 CKO, n=8 Littermate; Repeated measures ANOVA F (1, 16)=21.38, P=0.0003; F (1, 16)=9.902, P=0.0062. post hoc Bonferroni's test). (FIG. 4W) Immunoblot showing levels of GluN1 co-immunoprecipitated with EphB2 in the ipsilateral dorsal horn (DH) in cKO mice compared to wildtype littermate controls (C). A fraction of the same sample was immunoblotted for EphB2 (EphB2, IP). DH lysates were probed as indicated for EphB2 and GluN1.

FIGS. 5A-F. Ephrin stimulates VLK secretion, and the kinase activity of VLK is necessary to induce the EphB2-NMDAR interaction. (FIG. 5A) VLK mCherry lentivirus-infected cultured cortical neurons (DIV7-8) were treated with clustered EphrinB2 (+eB2) or control (C) reagents in ACSF for 45 min. RFP Trap agarose beads were used to immunoprecipitate conditioned ACSF and immunoblotted with a custom-generated RFP antibody (ACSF-IP, top). Conditioned ACSF (ACSF, middle) and cell lysates (Lysate) were probed with beta-actin as a control to show that the conditioned media was free of any intracellular protein. mCherry was probed as lysate control (Lysate, Bottom blot). (FIG. 5B) PKDCC expression in Control (C) and Ephrin-treated Real DRGx neurons. No expression is depicted as Zero (black), Low is 1 to 10 puncta of PKDCC, and High is above 10. (FIG. 5C) Percentage of PKDCC expressing neurons after Control or ephrin stimulation of real DRGx neurons. (FIG. 5D) Pkdcc mRNA expression in rat cortical neurons (Unpaired t-test, ***pvalue=0.001). (FIG. 5E) Representative images of PLA of EphB2-NMDAR after treatment with soluble recombinant kinases. HEK293T cells were transfected with GluN1, GluN2B, EphB2, and EGFP. Cells were treated with Control, ephrin-B2, active recombinant VLK, and VLK-KD proteins (as indicated) for 45 minutes before fixation. The upper panels show the surface PLA signal alone (magenta). The lower panels are the merged images with EGFP in green. Scale bar=10 μm. (FIG. 5F) Quantification of the PLA puncta number. PLA puncta numbers are quantified by counting the puncta per 100 μm² in EGFP⁺ cells and normalized to the Control. (One way ANOVA followed by Tukey's test, **p=0.0050, 0.008, *p=0.01, ****p<0.0001, n=70 fields from 7 repeats).

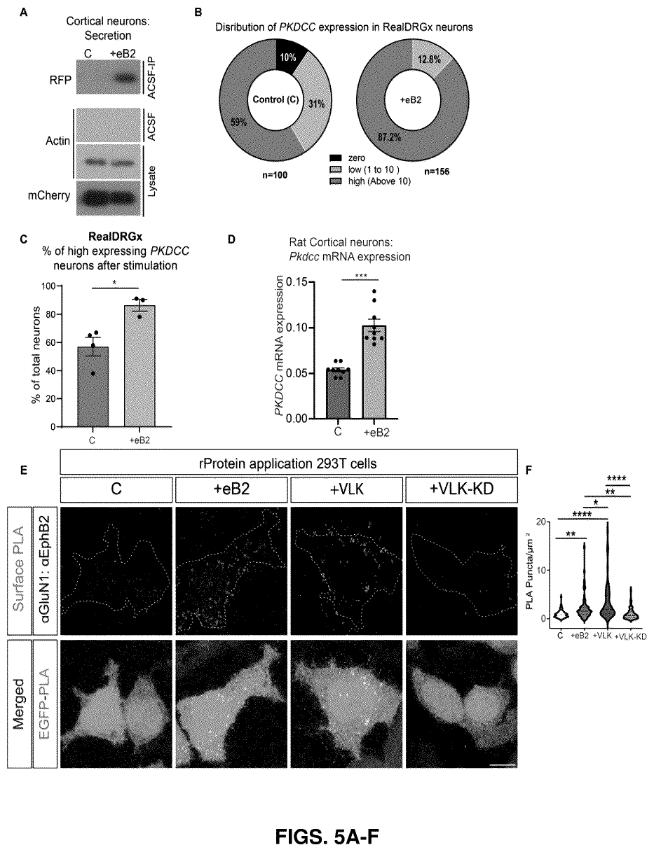

FIGS. 6A-C. VLK knockdown abolishes EphB2-NMDAR interaction in HEK 293T cells. (FIG. 6A) PKDCC mRNA expression in HEK 293T cells following treatment with Control (C) or PKDCC targeting shRNA 1,2 and 3 respectively, (One way ANOVA followed by Tukey's test, ****p<0.0001, ***p=0.0002, **p=0.0012, n=3) (FIG. 6B) Real-Time (RT) PCR-based quantification of relative mRNA levels of PKDCC (VLK), DIPK1C (Fam69C), and DIPK2B (Dia1R) from the Stable VLK KD cells (1) (−/−, grey bars) compared to control cells (+/+, black bars). (***p<0.001, unpaired t-test; n=3). (FIG. 6C) Immunoblotting for co-immunoprecipitated GluN1 with EphB2 from HEK293T cells transfected with Untreated (UT), Control (C), and VLK shRNA1 (1) (top blot). A fraction of the same EphB2 immunoprecipitated sample was immunoblotted for EphB2 (EphB2, IP). HEK293T cell lysates were probed as indicated for EphB2 and GluN1, and Tubulin (Lysate).

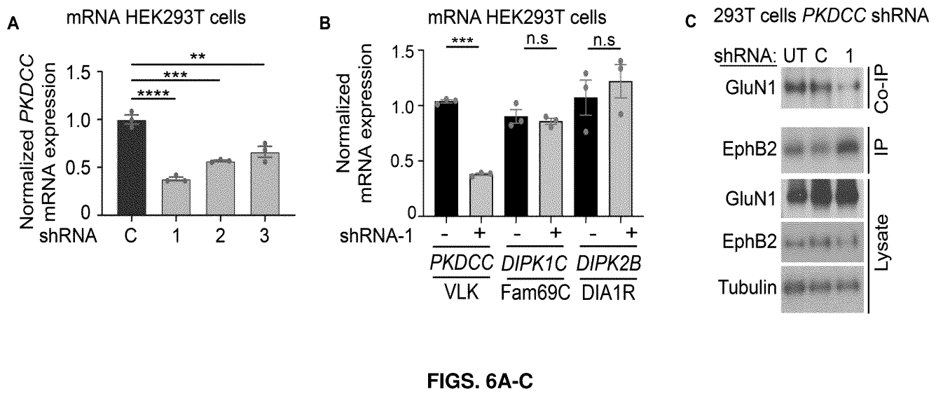

FIGS. 7A-E. VLK is enriched in the Synaptic vesicle fraction and not in the Dense Core Vesicles. (FIG. 7A) Western blots of Dense Core Vesicle purification fractions prepared from WT CD1 mouse brains showing VLK is not enriched in DCVs,but is enriched in Synaptic-like micro vesicles (SLMV). Post-nuclear supernatant (PNS), Post-mitochondrial supernatant (PMS), and Sucrose gradient fractions (1-10). Blots probed (from top to bottom): VLK, Synaptotagmin1, and Synaptotagmin 5 as DCV marker, flotillin as exosome marker, and GAPDH as a loading control. (FIGS. 7B-E) Parallel Reaction Monitoring (PRM) experiment confirming the presence of VLK (PKDCC) and corresponding tandem MS/MS spectra for 2 of the peptides isolated and monitored are shown. Extracted ion chromatograms for major MS/MS fragment ions from the two tryptic peptides studied are shown for (FIGS. 7B-C) peptide EMVLLER (SEQ ID NO: 1) at m/z: 445.2442(2⁺) male and female, respectively, and (FIGS. 7D-E) peptide SGQYLQNSTSSR (SEQ ID NO: 2) at m/z: 664.3155(2⁺) male and female, respectively.

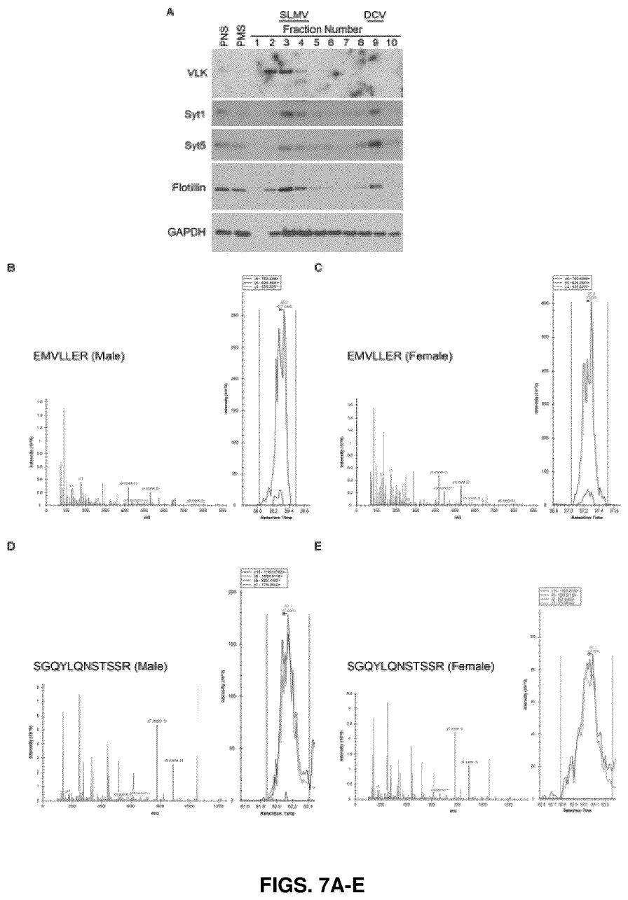

FIGS. 8A-H. VLK expression in mouse DRG and pain circuit. (FIG. 8A) Cell-type expression of Pkdcc in the sensory neurons of mouse DRG. Solid vertical lines separate major cell populations, and dashed lines show the separation into further subtypes. Data from Linnarssonlab.org/drg/ (FIG. 8B) Quantified representation of FIG. 8A delineating the fraction of each subtype displaying a positive expression of Pkdcc. Data from Linnarssonlab.org/drg/. (FIG. 8C) Pkdcc mRNA was detected in a significant number of DRG neurons (n=8, Pkdcc M=79.64, SEM=3.154; Nf200 M=67.15, SEM=1.156, Scn10a M=59.31, SEM=2.048). (FIG. 8D) Population distributions were similar between male and female mice. (FIG. 8E) Histogram showing the distribution and descriptive statistics of PKDCC expression in Nf200+ neurons. The area between the two dotted lines (1 and 10 puncta) indicates low Pkdcc-expressing neurons. (FIG. 8F) Sex-based analysis of Pkdcc expression in the Nf200 population shows no difference between males and females. (FIG. 8G) Histogram showing the distribution and descriptive statistics of Pkdcc expression in Scn10a⁺ neurons. The area between the two dotted lines (1 and 10 puncta) indicates Low PKDCC-expressing neurons. (FIG. 8H) Sex-based analysis of Pkdcc expression in the SCN10A population shows no difference between males and females.

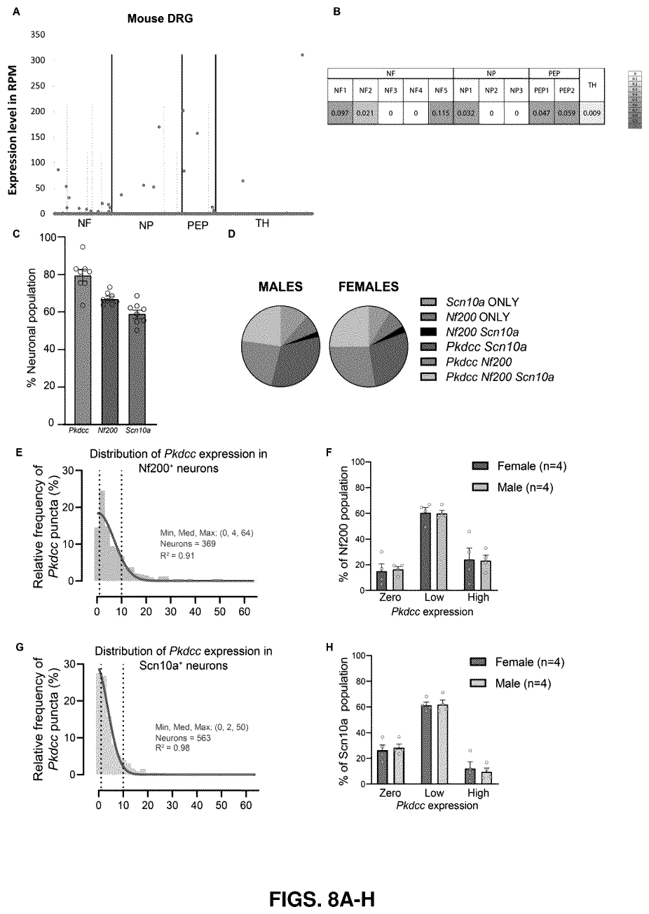

FIGS. 9A-D. VLK expression in human DRG. (FIG. 9A) The Size-distribution curve of PKDCC neurons shows greater expression in small to medium-diameter cells. (FIG. 9B) PKDCC expression and distribution across marker populations in the human DRG is similar across males and females. (FIG. 9C) Relative Fluorescence Unit (RFU) of bulk VLK expression in different human sensory tissues using the Somalogic system, showing significant detection of VLK across the DRG, Dorsal Horn (DH), Ventral Horn (VH), and spinal meninges. (FIG. 9D) Pkdcc mRNA expression in WT and cKO (Pirt-Cre) mice.

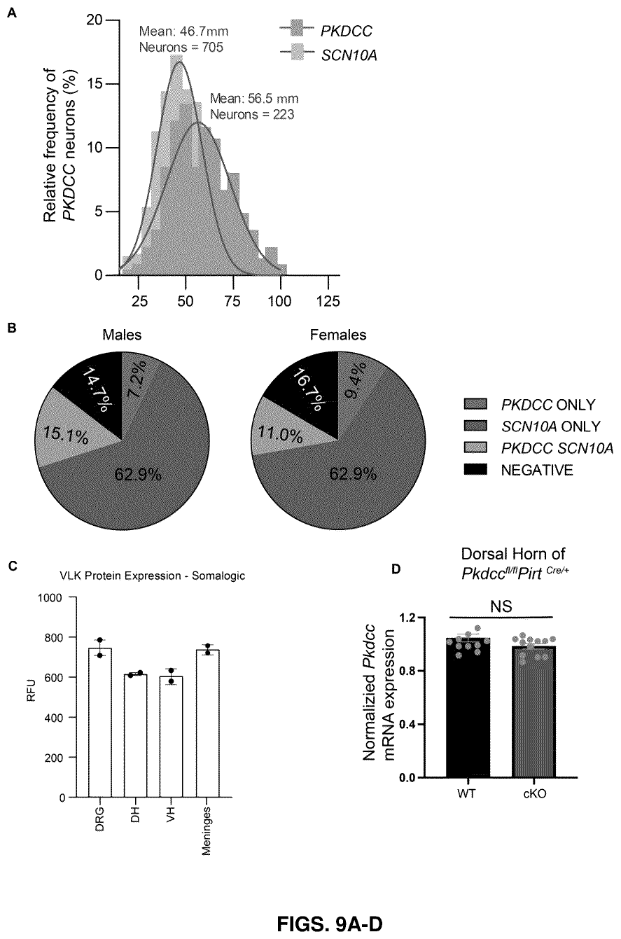

FIGS. 10A-C. Pkdcc is expressed in mouse lumbar spinal cord dorsal horn neurons. (FIG. 10A) RNAscope in situ hybridization with NeuN counterstaining was used to identify Pkdcc and Tacr1-expressing neurons. Scale bar=50 μm. The inset shows double-positive neurons indicated by the white borders. Scale bar=5 μm. (FIG. 10B) Percent of DH neurons expressing Pkdcc, Tacr1, and both markers. (n=8; Tacr1 M=18.25, SEM=2.644; Pkdcc M=41.00, SEM=3.140, Double Positive M=11.75, SEM=2.016). (FIG. 10C) A breakdown of expression in B by sex shows no significant difference between males and females (n=4 per sex; Two-way ANOVA F (1, 18)=4.141, P=0.0569.) DP=double positive.

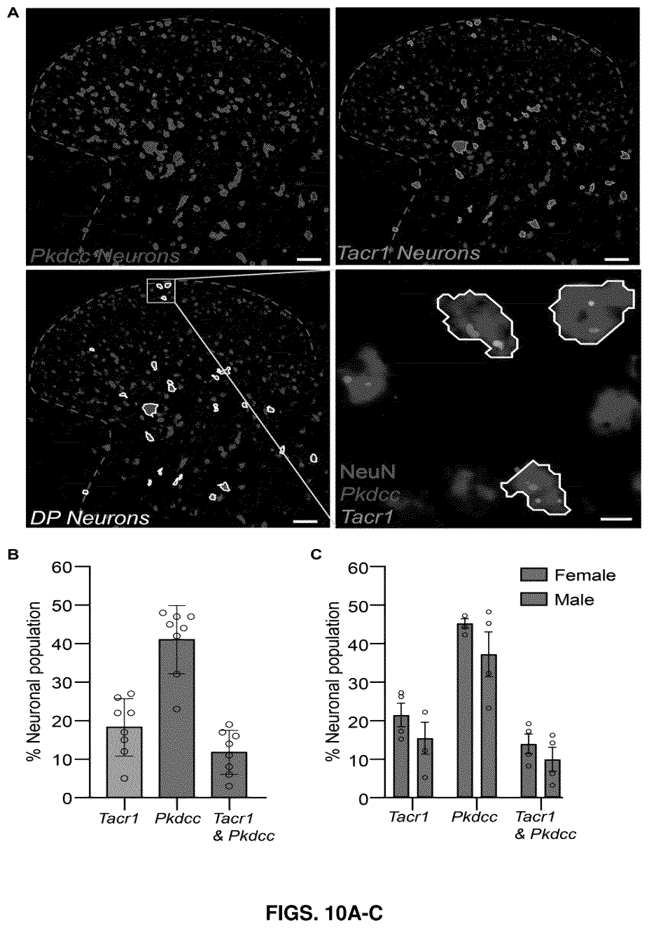

FIGS. 11A-D. PKDCC is expressed in the human lumbar spinal cord dorsal horn. (FIG. 11A) RNAscope in situ hybridization was used to identify PKDCC and TACR1-expressing neurons.Scale bar=50 μm. Inset shows superficial dorsal horn from FIG. 11A, the right panels display an example of a double positive neuron from the image. Scale bar=5 μm (FIG. 11B) Percent of PKDCC+ neurons across the DH lamina that also express TACR1. (FIG. 11C) Percentage of TACR1⁺ neurons that also express PKDCC. (FIG. 11D), a summary of total neurons counted for each donor (#1-3).

FIG. 12. Assay validation for inhibition of EphB2-NMDAR interaction by antibody blockade of VLK/PKDCC in cortical neurons. Untransfected cultured rat cortical neurons (7 DIV) were treated with ephrin-B2 or control reagents (Fc). Putative function blocking antibodies are pretreated for 1 hour prior to a 45 minute treatment with ephrin-B2. Following treatment lysates were co-immunoprecipited with anti-EphB2 antibodies and probed for GluN1 (N1, Top blot) to test for the interaction between EphB2 and GluN1 or with phospho-tyrosine (pY99) to test the for the kinase activation of EphB2 (Second blot). Inhibition of the interaction is indicated by a reduction in the intensity of the blot in the ephrin-B2 (eB2) lanes. Lower blots show lysates probe for EphB2 or GluN1 as labeled.

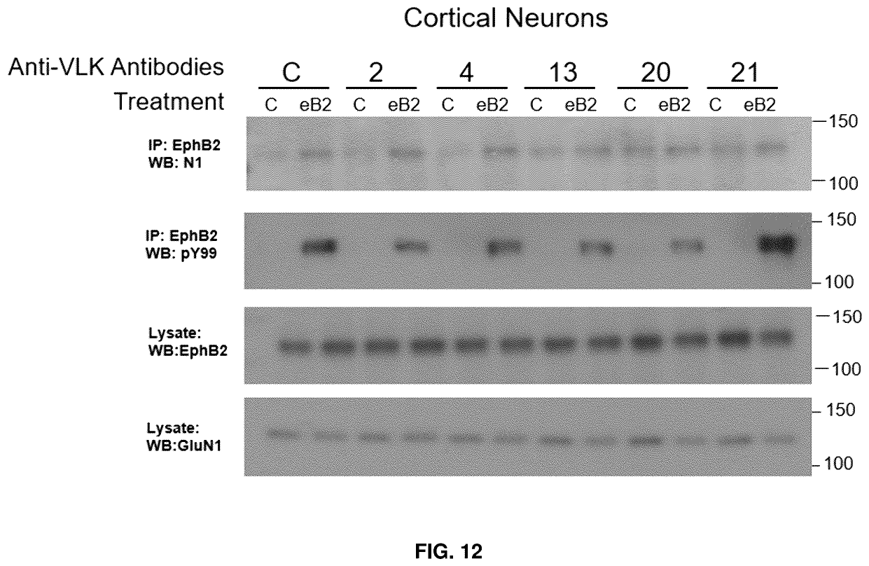

### DESCRIPTION OF ILLUSTRATIVE EMBODIMENTS

As discussed above, better understanding of the role phosphorylation events play in disease could be extremely important in developing new therapies. The research described here has uncovered that VLK/PKDCC, an ectokinase, is key in the regulation of synaptic plasticity and behavior through extracellular phosphorylation. One critical instance of its role is observed in the EphB-NMDAR interaction at excitatory synapses in the brain and spinal cord. The interaction, vital for normal NMDAR localization at synapses, is driven by ephrin-B ligand-mediated phosphorylation of EphB2 at the Y504 residue. Disruption of this interaction leads to the loss of NMDAR from synaptic sites. Further, the inventors' studies show that VLK secretion is ephrin-B dependent and crucial for the EphB2-NMDAR interaction. This reveals an intricate mechanism whereby extracellular phosphorylation by VLK regulates synaptic behaviors and pain responses.

The inventors have demonstrated that VLK, among the VLK/PKDCC family of tyrosine kinases, is uniquely capable of inducing the EphB-NMDAR interaction. This was evidenced by co-immunoprecipitation experiments in various neuronal models, including primary cortical neurons and iPSC-derived DRG sensory neurons. Additionally, VLK's role in pain modulation was underscored by the finding that its secretion, regulated by ephrin-B2, modulates synaptic interactions essential for pain perception.

The inventors' findings have also elucidated that VLK, localized to synaptic vesicles, is released from neurons in a SNARE-dependent fashion. This presynaptic release of VLK induces postsynaptic EphB2-NMDAR interaction, a novel mechanism in the regulation of synaptic protein-protein interactions. This mechanism is further supported by the observation that VLK is secreted from neurons upon ephrin-B2 activation, and its kinase activity is necessary for inducing the EphB2-NMDAR interaction. These findings are validated in human iPSC DRG neurons, in human tissue, and mouse and rat models.

This disclosure pertains to the discovery and application of extracellular kinases, particularly the Vertebrate Lonesome Kinase (VLK/PKDCC), in regulating synaptic protein-protein interactions and pain response mechanisms. This document describes tools and methods not only targeted at pain management, but also applications in other neurological disorders. For example, antibodies targeting specific sites and proteins can be designed to interact with sites such as EphB2 Y504, the protein VLK, and the GluN1 EphB2 binding site. In addition, drugs inhibiting VLK Kinase function will be formulated to suppress or alter the activity of the VLK kinase. Similarly, peptides that block VLK Kinase activation can prevent activation of the VLK kinase, including the inhibition of phosphorylation of Y504 on EphB2 and the GluN1 binding site for Y504. CRISPR and other genetic editing approaches for targeting expression of VLK/PKDCC can be utilized for the knockdown of VLK or the PKDCC gene, offering potential in modifying gene expression or function in various neurological conditions. Applications for these embodiments include acute pain, cancer pain, neuropathic pain, injury-induced pain, NMDAR dependent pain, and other types of pain linked to human suffering. Additionally, there are applications in neurological disorders such as Alzheimer's disease, schizophrenia, and stroke due to the importance of the EphB-NMDAR interaction in these disorders.

In sum, the inventors' research has uncovered that VLK/PKDCC is key in the regulation of synaptic plasticity and behavior through extracellular phosphorylation. One critical instance of its role is observed in the EphB-NMDAR interaction at excitatory synapses in the brain and spinal cord. The interaction, vital for normal NMDAR localization at synapses, is driven by ephrin-B ligand-mediated phosphorylation of EphB2 at the Y504 residue. Disruption of this interaction leads to the loss of NMDAR from synaptic sites. Further, these studies show that VLK secretion is ephrin-B dependent and crucial for the EphB2-NMDAR interaction. This reveals an intricate mechanism whereby extracellular phosphorylation by VLK regulates synaptic behaviors and pain responses. These and other aspects of the disclosure are described in detail below.

### I. Vertebrate Lonesome Kinase (VLK)

Vertebrate Lonesome Kinase (VLK), also known as Protein Kinase Domain Containing, Cytoplasmic (PKDCC), is a Secreted tyrosine-protein kinase that mediates phosphorylation of extracellular proteins and endogenous proteins in the secretory pathway, which is essential for patterning at organogenesis stages. It mediates phosphorylation of MMP1, MMP13, MMP14, MMP19 and ERP29 and probably plays a role in platelets: rapidly and quantitatively secreted from platelets in response to stimulation of platelet degranulation. It may also have serine/threonine protein kinase activity. It is required for longitudinal bone growth through regulation of chondrocyte differentiation and may be indirectly involved in protein transport from the Golgi apparatus to the plasma membrane.

PKDCC_HUMAN, Q504Y2 (Protein Symbol: Q504Y2-PKDCC_HUMAN) is called Extracellular tyrosine-protein kinase PKDCC with Protein Accession No. of Q504Y2 and secondary accessions of D6W5A0 and Q96109. It has 493 amino acids and a molecular mass of 54132 Da. It is most highly expressed in NK cells, heart, saliva and pancreatic juice.

A variety of antibodies to VLK are available from LSBio (SGK493), OriGene Antibodies (TA306903; TA358286), Invitrogen, Novus Biologicals Antibodies, Sino Biological, Boster Bio Antibodies (A11391), Santa Cruz Biotechnology (SCBT) Antibodies (G-10), and Rockland (Anti-VLK RABBIT Antibody-600-401-FX0).

This disclosure focuses on various aspects of VLK's biological roles, mechanisms of action, and potential therapeutic applications. Mass spectrometry analysis of protein extracellular domains by other revealed extensive phosphorylation events on specific serine, threonine, and tyrosine residues. Similarly, researchers have shown that phosphorylation, mediated by two kinase families, has significant but underexplored implications in the nervous system. Indeed, FAM20C mutations have been connected to Raine syndrome, highlighting the importance of extracellular kinases in neurological disorders. VLK also is essential for mouse survival, indicating its critical role in neural development and function.

VLK's role in the EphB-NMDAR interaction at excitatory synapses across the brain and spinal cord is crucial. Ephrin-B ligand-mediated phosphorylation of EphB2 at the Y504 residue in the Fibronectin-type III domain is essential for normal NMDAR localization at synapses. Disruption of this interaction results in NMDAR loss from synaptic sites, affecting synaptic plasticity and behavior. VLK secretion, regulated by ephrin-B2, is pivotal for the EphB2-NMDAR interaction, establishing a direct link between VLK activity and synaptic function.

VLK is secreted in a SNARE-dependent manner and is found in synaptic vesicles, indicating its active role in synaptic transmission. The presynaptic release of VLK is necessary for postsynaptic EphB2-NMDAR interaction, a novel finding in synaptic protein-protein interaction regulation. VLK's role in pain modulation was further underscored through knockout studies, where its absence rendered mice impervious to post-surgical pain without affecting normal nociception.

Research extended to human-induced pluripotent stem cell (iPSC)-derived dorsal root ganglion (DRG) sensory neurons and spinal cord synapses. The study involved assessing the phosphorylation of Y504 on EphB2 in the extracellular space, a process requiring secreted protein kinases. VLK's role was further confirmed through experiments using human tissues, where ephrin-B2 stimulation released a soluble, protein-based activity phosphorylating the EphB2 ectodomain at Y504 in an ATP-dependent manner.

VLK, among the VLK/PKDCC family of tyrosine kinases, uniquely induces the EphB-NMDAR interaction. In-depth analysis involved various neuronal models, including cell lines, primary cortical neurons, and human iPSC-derived neurons. VLK co-localizes with synaptic vesicle markers, suggesting its presynaptic localization and involvement in synaptic vesicle dynamics. The secretion of VLK from neurons is critical for inducing synaptic interactions essential for pain perception and management.

### II. Formulation and Administration

The present disclosure provides pharmaceutical compositions. Such compositions comprise a prophylactically or therapeutically effective amount of an agent, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the agent, preferably in purified form, together with a suitable amount of carrier so as to provide the form for proper administration to the patient. The formulation should suit the mode of administration, which can be intracranial, to the dorsal ganglia, into spinal fluid, oral, intravenous, intraarterial, intrabuccal, intranasal, nebulized, bronchial inhalation, intra-rectal, vaginal, topical or delivered by mechanical ventilation.

Pharmaceutically acceptable salts include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

### III. Applications

As discussed above, VLK is identified as a pivotal component in the presynaptic release mechanism within dorsal spinal cord neurons. Its secretion, regulated by ephrin-B2 stimulation, is SNARE-dependent and crucial for direct protein-protein interactions at synapses. The present disclosure demonstrates that nociceptor-specific knockout of VLK renders mice resistant to post-surgical pain without altering normal nociception. This indicates VLK's essential role in pain modulation following injury. In addition, VLK's secretion is necessary for the EphB2-NMDAR interaction, crucial for synaptic localization of NMDAR. This interaction is modulated through VLK-mediated extracellular phosphorylation of EphB2, specifically at the Y504 residue.

VLK's interaction with synaptic vesicle marker proteins, its presence in the synaptic vesicle fraction, and its trafficking in axons form the basis of its synaptic function. The data presented here show VLK is integral to synaptic plasticity, potentially offering targets for non-opioid pain therapies and insights into the regulation of synaptic behavior. These observations extend beyond pain management, indicating VLK's broader role in nervous system development, viability, and neuronal functionality. Indeed, the inventors' findings open avenues for developing therapeutic interventions targeting VLK/PKDCC for pain management, acute pain, cancer pain, neuropathic pain, injury-induced pain, NMDAR dependent pain, and other types of pain linked to human suffering. Additionally, their application in neurological disorders such as Alzheimer's disease, schizophrenia, NMDAR encephalitis, autism spectrum disorder, and stroke due to the importance of EphB-NMDAR interaction in these disorders.

### IV. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials & Methods

Animals. The Institutional Animal Care and Use Committee of Thomas Jefferson University, Tulane University, and UT Dallas approved all animal procedures. All behavior tests were conducted during the light hours of the cycle, between 6:00 a.m. and 6:00 p.m. Animals were group-housed, with each cage having an equal balance of treatment conditions/genotypes. Dissociated cortical culture neurons were prepared from E17-18 Long Evans rat (Charles River). Wild-type CD-1 mice (Charles River) and transgenic mice (generated for and described in this study) were housed (3-5 mice per cage) in Thomas Jefferson University, Tulane University, and UT Dallas laboratory animal facilities.

Human Tissue. The Institutional Review Boards of UT Dallas (UTD) reviewed and approved protocols. All experiments conformed to relevant guidelines and regulations. Dorsal root ganglion tissues were collected from the donors after neurological determination of death within 2-3 hours of cross-clamp. Donor information is provided in Table S1.

HEK293T cell culture and transfection. HEK293T cells were cultured and transfected using calcium phosphate as described (4). NMDAR-transfected HEK293T media was supplemented with 50 μM APV (Tocris Bioscience) and 10 μM MK801 (Tocris Bioscience) to prevent excitotoxicity (4). Stable 293T VLK knockdown cell line was generated by transfecting cells with a shRNA targeting PKDCC (5′-CCCAACGTGCTGCAGCTCT-3′; Horizon Discovery Ltd.) followed by prolonged puromycin (2 μg/ml) selection over 10 passages. The stable PKDCC knockdown clone was maintained in puromycin (0.5 μg/ml) containing media.

Primary neuronal culture and transfection. Primary neuronal cultures were derived from embryonic day 17-18 (E17-18) rat cerebral cortex and maintained as described previously (4, 6). 8×10⁶ neurons were plated for 10 cm dishes and 1×10⁶ neurons were plated per well of 6 well dishes for biochemical assays. 2×10⁵ neurons were plated on glass bottom dishes for live cell imaging experiments. As previously described, neuronal cultures were transfected using Lipofectamine 2000 (4, 6).

Human iPSC neuronal differentiation and maturation (hiSNs). Human iPSC-derived immature sensory neurons (RealDRGx™) were produced by Anatomic Inc. with the Anatomic SensoX-DM kit (Anatomic #7007). Neurons at day 7 post-differentiation were plated on glass coverslips coated with poly-L-ornithine (Sigma, Castle Hill Australia) and Matrix 3 (Anatomic Inc., Minneapolis, USA) in 96-well tissue culture plates (Corning) at a density of 40,000 cells/cm², and maintained in Chrono™ Senso-MMx1 maturation medium (Anatomic cat #7008) for 3 days followed by Chrono™ Senso-MMx2 for the remaining 28 days (DIV28). Growth media was exchanged three times a week, and stimulations were performed at DIV28.

Expression Constructs. Myc-GluN1, GluN2B, FLAG-EphB2, mutant FLAG-EphB2 (Y504E and Y504F), and EGFP have been previously described (4, 6). Syp1-EGFP and mTurquoise2 were previously described (33, 34). Flag-tagged constructs of VLK, VLK-Kinase dead (KD), DIA1, DIA1R, Fam69A, Fam69B, and Fam69C have been described (1, 35). The Flag-tagged VLK rescue construct (shRNA resistant) was created by using site-directed mutagenesis (Stratagene, La Jolla, CA) to convert the PKDCC shRNA target site described above to 5′-CCaAAtGTaCTaCAaCTa 3′ (SEQ ID NO: 3) with the following primers Fwd 5′-CTGCTGGAGCGGCTGCGGCACCCAAATGTACTACAACTATATGGCTACTGCTACCAG GAC-3′ (SEQ ID NO: 4) and Rev 5′-TCCTGGTAGCAGTAGCCATATAGTTGTAGTACATTTGGGTGCCGCAGCCGCTCCAGC AG-3′ (SEQ ID NO: 5) without changing the protein sequence. The VLK-mCherry lentiviral construct was generated by PCR amplifying VLK from above and subcloning it downstream of the human synapsin promoter to replace NSG2 in pFCK-NSG2-mCherry (36) using standard restriction cloning.

Lentivirus production and transduction. Lentivirus was packaged using a second-generation packaging system by transfecting HEK293T cells with VLK-mCherry, psPAX2, and pMD2.G as previously described (36). Lentivirus was harvested from transfected cell media by ultracentrifugation 0.45 μm filter-sterilized supernatant at 110,000×G for 2h. DIV3 neurons were transduced with VLK-mCherry lentivirus (MOI 2-3) and assayed at DIV7 for ephrinB2-dependent VLK secretion.

Western Blotting, Immunoprecipitation (IP) and Co-IP. HEK293T cells and cultured neurons were lysed in RIPA buffer to generate protein lysates. Cortices of postnatal day 35 (P35) transgenic mice Pkdcc^{flox/flox} CaMKII^{Cre/+} and its WT littermate controls were also lysed using RIPA buffer and an equal amount of protein was used to perform IP. Pooled dorsal horns from Pkdcc^{flox/flox} Pirt^{Cre/+} uninjured mice and their WTlittermate controls were lysed in RIPA. Following lysis, an additional Percoll gradient spin was carried out to remove myelin and unwanted cellular debris (37) and an equal amount of protein was used to perform IP. For conditioned ACSF IP, ACSF was filtered through a 0.22 mm syringe filter (Millipore, cat #SLGV004SL) and incubated with antibody for 2 h at 4° C. followed by addition of Protein-G beads and incubation overnight at 4° C. IP, Co-IP, and Western blotting were performed as previously described (4, 6). RFP Trap agarose beads (ChromoTek, cat #rta) were used to IP VLK-mCherry from as per manufacturer's instructions. For RFP Trap IP conditioned ACSF was filtered as described above and further modified by addition of a final concentration of 10 mM Tris pH 7.5, NaCl 150 mM, 0.5 mM EDTA and 0.5% NP-40. Antibodies used for IP were mouse monoclonal Anti-FLAG M2 (Sigma, cat #F1804, lot #SLCN3722), goat polyclonal anti-EphB2 (R&D Systems, cat #AF467, lot #CVT0315041), mouse monoclonal anti-pTyr (PY99) (Santa Cruz, Cat #SC-7020; lot #L022)

Cellular Stimulations. Ephrin-B2-Control or clustered ephrin-B2 treatments were performed as previously described (9). Briefly, control IgG-Fc (Cat #110-HG-100; R&D Systems) and recombinant ephrin-B2-Fc (Cat #7397-EB-050; R&D Systems) were clustered with Donkey anti-Human IgG (Cat #709-005-149; Jackson ImmunoResearch) for 30 min at room temperature. Multimerized control and ligand were mixed in 37° C. artificial cerebrospinal fluid (ACSF, 140 mM NaCl, 5 mM KCl, 1 mM MgCl2, 2 mM CaCl2, 20 mM glucose, and 10 mM HEPES, pH 7.3) and applied on cells for 45 min. The ACSF was collected from the wells to detect secreted VLK, passed through a 0.22 μm filter, and immediately subjected to IP as indicated or stored at −80° C. until used.

Recombinant protein—HEK293T cells were treated with 100 ng recombinant VLK, Fam69C, or DIA1. Synaptosomes were treated with 100 ng recombinant VLK. 1 mM ATP (Cat #A9187; Sigma) was applied as indicated. Recombinant PAP treatment was applied at 100 ng.

Botulinum toxin—Active Botulinum toxin A or B (BoNTA/B) (Cat #128C and 138B (discontinued; List labs, CA) were applied at 100 pM in ACSF. Cells were pretreated for 1 h with toxins in ACSF followed by application of ACSF containing toxins with either control or ephrin-B2 for 45 min.

Proximity Ligation Assay (PLA). As previously described, PLA experiments on HEK293T cells, mouse brain, and human spinal cord synaptosomes were performed (4). Synaptosome PLA combined with vGlut1 immunostaining was also previously described (4).

In vitro Kinase assay. For the in vitro kinase assay purified recombinant protein fragment with EphB2 extracellular domain (EphB2 (200 ng), active; Millipore) and active VLK (100 ng), VLK-KD (100 ng), Fam69C (100 ng), DIA1 (100 ng) was used. The kinase reaction was started by adding 10 mM MnCl2, 10 mM MgAc, and 100 μM ATP in ACSF buffer. After 30 min of incubation at 37° C., phosphorylated proteins were separated by SDS-PAGE and analyzed by western blotting with anti-phosphotyrosine antibody (PY99) and EphB2. This protocol was also previously described (6)

RNA extraction and qPCR. Total RNA from HEK293T cells was extracted using TRI reagent (Cat #TR118; Molecular Research Center Inc.) as per the manufacturer's instruction. Total RNA from mice cortices was extracted using the Direct-zol RNA microprep kit (Cat #R2062; Zymo Research) following the manufacturer's instructions. RNA was quantified using a Nanodrop and 1 ug of total RNA was used to prepare cDNA using qScript cDNA Synthesis kit (Cat #90547-100; Quantabio) as per manufacturer's instructions for quantitative polymerase chain reaction (qPCR). PCR was set in triplicates using SYBR green (Applied Biosystems) in a 20 μL volume and using 15 ng cDNA template. PCR was run using the StepOne Plus system, and the quantification was performed using the comparative Ct (ΔΔCt) method. The qPCR primers used are described in Table S2.

VLK, Fam69C, DIA1, and VLK-KD Protein Purification. GST-tagged kinase-containing plasmids were transformed into competent BL21 cells. A single colony was picked and grown overnight in 5 mL LB-ampicillin starter culture. 1 Liter LB-ampicillin cultures were inoculated and grown at 37° C. and 250 rpm until OD600 reached 0.8-1. Cultures were cooled to 16° C. 0.03 mM IPTG (optimized for VLK) was added. The cultures were induced overnight at 16° C. A 2 mL glutathione resin slurry was loaded into a column and washed with 5 mL resuspension buffer by gravity flow. The column was stored in a resuspension buffer at 4° C. for next-day use. The following day, the bacteria were pelleted at 6000 rpm for 10 minutes, and the pellet was resuspended in 35 mL resuspension buffer. Lysozyme and PMSF were added and mixed. Lysate was incubated on ice for 10 min. Lysate was sonicated 20×3 sec ON, 3 sec OFF on ice using a probe sonicator. Lysate was centrifuged at 15,000 rpm (˜28,000×g) for 20 min at 4° C. The supernatant was transferred to a clean container immediately. The supernatant was loaded slowly onto the glutathione column. The column was washed 4× with wash buffer, 5 mL each. The column was plugged, and 4 mL elution buffer was added. Beads were suspended and incubated for 10 min. The 4 mL elution was collected, and another 2 mL elution buffer was added to the column and collected. Protein elution was then dialyzed overnight.

Brain Fractionation. Synaptosomes—Synaptosomes from wild type CD-1 mice brains were prepared and plated as previously described (4, 6).

Post-synaptic Density (PSD) fractionation—PSD fractions were prepared from postnatal day 21 (P21) wild-type CD-1 mice (38). Brains were homogenized on ice in 0.32 M sucrose, 4 mM HEPES, pH 7.4, containing a protease inhibitor cocktail (Sigma) and 1 mM PMSF. After removing the nuclear fraction (P1) by centrifugation at 1,000×g for 15 minutes at 4° C., non-synaptic fractions were further centrifuged at 10,000×g at 4° C. to obtain the crude synaptosomal fraction (P2). This pellet was resuspended in 10 volumes of HEPES-buffered sucrose and then spun again at 10,000×g for another 15 min. The resulting pellet was lysed by hypo-osmotic shock in water, rapidly adjusted to 4 mM HEPES pH 7.4, and mixed constantly for 30 min at 4° C. The lysate was centrifuged at 25,000×g for 20 min and the pellet (P3) was resuspended in HEPES-buffered sucrose. The crude synaptic vesicle supernatant (S3) was saved for further purification of synaptic vesicles. The resuspended membranes were carefully layered on a discontinuous gradient containing 0.8 to 1.0 to 1.2 M sucrose and centrifuged at 150,000×g for 2h. Synaptic plasma membranes were recovered in the layer between 1.0 and 1.2 M sucrose and diluted to 0.32 M sucrose by adding 2.5 volumes of 4 mM HEPES pH 7.4. Membranes were pelleted by centrifugation at 150,000×g (36,000 rpm in SW50.1) for 30 minutes. Pellet (SPM) was resuspended in 3-5 ml of ice-cold 50 mM HEPES pH7.4, 2 mM EDTA, plus protease/phosphatase inhibitors. Triton X-100 was added to 0.5%. Samples were rotated in the cold room for 15 min. Samples were centrifugated at 32,000×g (16,318 rpm in SW50.1) for 20 min to obtain the PSD-1T pellet. Pellet was resuspended in 3 mL ice-cold 50 mM HEPES pH7.4, 2 mM EDTA plus protease/phosphatase inhibitors). 1 mL was saved as PSD-1T. To half of the remaining resuspended pellet (˜1 mL), Triton X-100 was added to 0.5% and rotated in the cold room for 15 minutes. Sample was centrifugated at 200,000×g (41,000 rpm in SW50.1) for 20 min to obtain the PSD-2T pellet. In a separate experiment, resuspend the second half of the PSD-1T pellet and incubate for 10 min in ice-cold 3% sarcosyl (N-lauroyl sarcosine) in 50 mM HEPES pH7.4, 2 mM EDTA, plus protease/phosphatase inhibitors. Centrifuge at 200,000×g (41,000 rpm in SW50.1) for 1 hour to obtain the PSD-S pellet. Pellets were resuspended in 50 mM HEPES pH7.4, 2 mM EDTA plus protease/phosphatase inhibitors.

Dense Core Vesicles Isolation (DCV)—DCVs were prepared from postnatal day 21 (P21) wild-type CD-1 mice (39). Brains were homogenized on ice in a homogenization medium (0.26 M sucrose, 5 mM MOPS, and 0.2 mM EDTA). Homogenate was then spun at 4000 rpm (1000×g) for 10 min at 4° C. in a fixed-angle microcentrifuge to pellet nuclei and larger debris. The post nuclear supernatant (PNS) was collected and spun at 11,000 rpm (8000×g) for 15 min at 4° C. to pellet mitochondria. The post-mitochondrial supernatant (PMS) was then collected, adjusted to 5 mM EDTA, and incubated for 10 min on ice. A working solution of 50% OptiPrep (iodixanol) [five volumes of 60% OptiPrep (one volume of 0.26 M sucrose, 30 mM MOPS, and 1 mM EDTA)] and homogenization medium were mixed to prepare solutions for discontinuous gradients in Beckman SW 55 tubes: 0.5 ml of 30% iodixanol on the bottom and 3.8 ml of 14.5% iodixanol, above which 1.2 ml of EDTA-adjusted PMS was layered. Samples were spun at 45,000 rpm (190,000×g) for 5 hours. 10 fractions were collected and analyzed by Western blot. A clear white band at the interface between the 30% iodixanol and the 14.5% iodixanol was the DCV sample (fraction 9 of Western blot).

Proteomic analysis. Proteins samples from S3 crude synaptic vesicle fraction from 3 male and 3 female mice were concentrated by short SDS-PAGE runs into single-protein bands before tryptic digestion and peptide extraction. Peptides were desalted with Empore C18 High Performance Extraction Disks (40), and the eluted peptide solutions were partially dried under vacuum and then analyzed by LC-MS/MS with a Thermo Easy nLC 1000 system coupled online to a Q Exactive HF with a NanoFlex source (Thermo Fisher Scientific) as previously described (41). All data were analyzed by MaxQuant proteomics software (version 1.5.5.1) with the Andromeda search engine (42, 43) using a mouse protein FASTA (mouse [Mus musculus] protein database; Uniprot; Reviewed, 21,989 entries [06182020]). Additionally, targeted mass spectrometry analysis was carried out to identify extracellular tyrosine-protein kinase (VLK) in both female and male preparations. Theoretical monoisotopic masses of 6 tryptic peptides of VLK that are commonly seen and documented in the Global Proteome Machine resource (GPM, (44)) were used in an inclusion list. The resulting raw mass spectrometry files were imported into Skyline proteomics software (45), and the presence of precursor and transition ions confirmed to identify VLK in both male and female peptide pools unequivocally.

Live cell imaging, Immunocytochemistry and Image analysis. Live imaging was carried out on rat cortical neurons plated on 35 mm glass bottom dishes (Cell E&G). DIV17 cultured rat cortical neurons were co-transfected with VLK-mCherry, Syp1-EGFP, and mTurquoise2. Live cell imaging was carried out on DIV21-22 neurons by replacing cell media with 37° C. ACSF for the duration of live imaging. Time-lapse images of live neurons were acquired on the Leica SP8 using a 63× objective lens. Images of neuronal axons (determined by the mTurquoise cell fill) were acquired at 2 frames/sec for 5 min as a single focal plane. Laser power was kept at low levels to avoid photobleaching. After live imaging, cells were immediately fixed with 4% PFA/2% sucrose for 8 minutes for posthoc immunocytochemistry as previously described (4). PSD95 primary antibody was applied overnight at 4° C. PSD95 signal was detected with an AF647 conjugated secondary antibody. Permanent markers of different colors were used on the glass dish for reference of reimaging. Additionally, the location of each imaged axon was recorded using a computer-controlled stage. These recorded references were used to re-locate the same axons reimaged to visualize PSD95, VLK-mCherry, and Syp1-EGFP (Posthoc). Posthoc fixed cell confocal images were acquired as Z stacks at 0.4-0.6 mm intervals.

Images analyzed using Fiji (ImageJ). A segmented line was drawn along the axon segments to measure axon length and generate kymographs using the reslice tool. On the kymographs, puncta were categorized into 2 groups-Stationary and Moving. Moving puncta were distinguished as tilted straight lines. Stationary puncta were vertical straight lines that did not deviate from the first to the last frame of live imaging. Stationary, moving, and colocalized puncta were manually scored. The number of puncta in each category was divided by the total length of the axon to derive puncta density. For posthoc analysis, images were background subtracted, gaussian blurred, and registered with live images using Turboreg (33, 34). The straightened axon segments from posthoc image were added as a new frame to the live image after the last frame of the identical axon segment from live imaging to assess and determine which stationary puncta were maintained after fixation. The stationary VLK-mCherry and Syp1-EGFP puncta maintained after fixation were manually scored. A custom macro (4, 34) was used to determine colocalization of maintained puncta with each other and PSD95. The analysis did not include Axon segments with no stationary puncta after fixation. Moving VLK puncta velocities were determined by manually tracking moving puncta using the MTrackJ plugin.

DRG dissection, RNAScope, Immunohistochemistry. DRG from Mouse and Human donors were processed as previously described (46). Immunohistochemistry and RNAScope were carried out on mouse and human DRG as previously described (46). Briefly, mRNA transcripts were detected using the RNAscope Fluorescent Multiplex Assay (Advanced Cell Diagnostics) and RNAscope Fluorescent Multiplex Reagent Kit (#320851 & #323100). The RNAscope catalogue probes used to detect were (For mouse Pkdcc (#516961), Scn10a (#426011-C2), Tacr1 (#428781-C3) and human PKDCC (#525121), SCN10A (#406291-C2)). Counterstaining for NF200 and NeuN was done with immunohistochemistry on the same tissues. Tissues were blocked in 0.1M Phosphate Buffer with 10% Normal Goat Serum and 0.3% Triton-X. Primary antibody was incubated overnight at 4° C. Secondary antibodies were incubated for 1 hour at room temperature. Coverslips were mounted with Prolong Gold Antifade (Fisher #P36930). Images were taken on an Olympus Fluoview FV1000 confocal microscope at 20× magnification. Histological analyses were carried out using Olympus Cellsens software. Frequency distribution, colocalization data and diameter measurements were calculated as described before (47). The Cellsens Count and Measure tool was used to conduct colocalization and cell area analyses in the DRG and spinal cord. To determine the expression-based distribution of PKDCC transcripts in the DRG neurons, expression levels were defined as Low (1-10 puncta) and High (10+ puncta).

cFos and NeuN staining of free-floating tissue sections. Adult mice were euthanized by isoflurane inhalation. Lumbar spinal cords were dissected and drop fixed in 10% formalin for 2 hours, cryoprotected, embedded in OCT (Fisher #1437365) and frozen on dry ice. Tissues were cryostat sectioned at 40 mm. Tissues were blocked in 0.1M Phosphate buffer with 10% Normal Goat Serum and 0.3% Triton-X and incubated in cFos and NeuN primary antibodies for 48 hours at 4° C. Secondary antibodies were incubated for 2 hours at room temperature. Sections were slide mounted and cover slipped with Prolong Gold Antifade. Images were taken on an Olympus Fluoview FV1000 confocal microscope at 20× magnification. cFos⁺ cell counts were carried out using Imaris software.

Generation of a VLK-specific Antibody. Antibodies against the ATP binding region of VLK were generated in rabbits against a peptide of the sequence Ac-CKALKAVDFSGHDLGS-NH₂ (SEQ ID NO: 6) (Covance, Denver, PA). Serum was collected and passed through a column containing SulfoLink Coupling Resin (Thermo Scientific) conjugated to KLH peptide. The serum was then incubated overnight in a column containing resin conjugated to the above VLK peptide. The antibody was eluted with glycine pH 2.0 and then extensively dialyzed in PBS.

Somascan Assay. Tissue lysates were prepared from fresh-frozen DRG, and from the dorsal and ventral horn portions of the lumbar spinal cord. The tissues were placed in T-PER Tissue Protein Extraction Reagent (Thermo Scientific, Cat #78510) with additional 1× Halt Protease Inhibitor Cocktail (Thermo Scientific, Cat #87786) and homogenized using Precellys Soft Tissue Homogenizing beads (Bertin Corp, Cat #P000933-LYSKO-A.0). Samples were centrifuged at 14,000×g for 15 minutes in the cold room. The resulting supernatant was quantified Micro BCA™ Protein Assay Kit (Thermo Scientific, Cat #23235) and normalized accordingly. Proteins were profiled using the SOMAScan platform. 7000 analytes were measured on the SOMAScan assay. The quality controls were performed by SomaLogic to correct technical variability within and between runs for each sample.

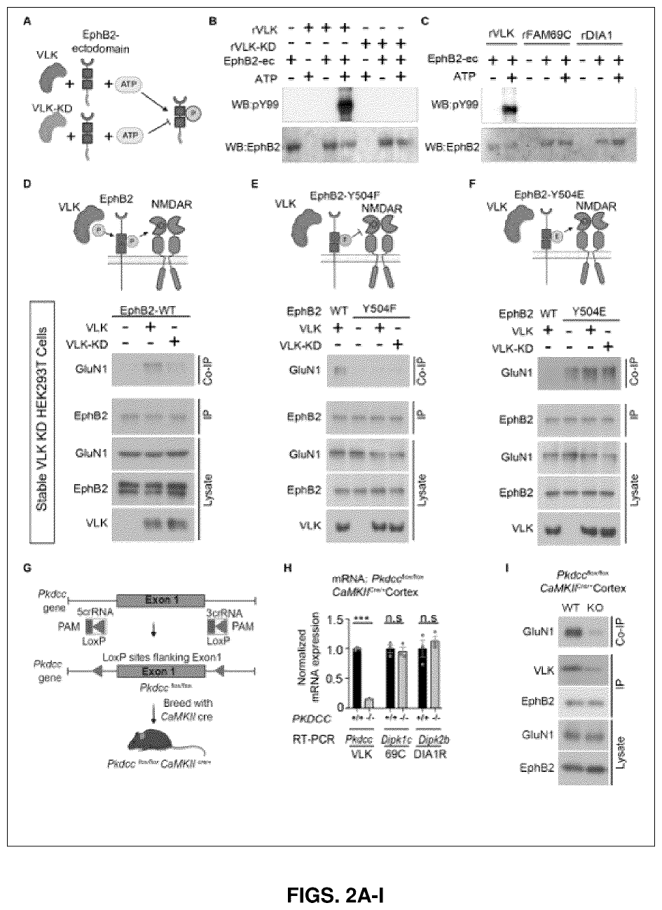

Generation of Pkdcc^{foxl/flox} mice and respective cKO mice lines. Pkdcc-floxed mice were generated at the UT Health San Antonio Mouse Genome Engineering Facility via CRISPR/Cas9-mediated HDR in zygote. Briefly, two DSBs flanking the first exon and upstream enhancer sites of the Pkdcc gene were produced and LoxP casettes were introduced via homologous recombination (shown in FIG. 2).

The recombination induced by Cre-recombination of exon 1 would result in causing Pkdcc loss of function. Thus, resulted mice were bred, and homozygous Pkdcc^{flox/flox} was selected by PCR genotyping. For genotyping, DNA was extracted from the ear samples during weaning to determine the genotypes of Pkdcc flox mice (KAPA mouse genotyping kit). The primers used for genotyping were mentioned in Table S2. The PCR products were visualized by electrophoresis on a 2-3% agarose gel. The Pkdcc floxed mice were bred with CaMKII-Cre mice to delete Pkdcc in excitatory neurons. The Pirt-Cre transgenic mouse line in this study was obtained from Xinzhong Dong at Johns Hopkins University (C13783-Pirt-Cre mice) and used to generate sensory neuron-specific knockout of Pkdcc. To determine if VLK is acting on EphB2 receptors expressed on these neurons, the inventors generated a mouse line expressing LoxP sites flanking the third exon of the Ephb2 gene. These mice were crossed with the Tacr1-Cre driver line to create conditional knockouts of EphB2 in spinal cord projection neurons.

Behavior. Mechanical Withdrawal Thresholds were determined using von Frey Filament testing, following the method of (48). Animals were habituated to plexiglass chambers (11.4×7.6×7.6 cm) suspended on wire mesh racks. Baseline withdrawal thresholds were measured before experimental treatments, and subsequent testing was done at various timepoints indicated in each figure. Mechanical sensitivity was determined by applying von Frey filaments to the plantar aspect of the hind paws, and a response was indicated by flicking or withdrawal of the paw. For mice subjected to hindpaw incision surgeries, filaments were instead applied to the heel of the hindpaw near the site of injury but avoiding scar tissue.

Mouse grimace scoring was conducted using the Mouse Grimace Scale (MGS) described by (49). After acclimating to the suspended plexiglass chambers for an hour, mice were scored according to the MGS by blinded experimenters at baseline and post treatment time points which are indicated in each figure. The grimace scores were averaged by group at each time point and plotted respectively.

Intrathecal injections were performed as previously described under isoflurane anesthesia (6). Drugs were administered in a 5 μL volume with a Hamilton syringe. Drugs were diluted in sterile saline. Heat denaturation was done by heating proteins at 70° C. for 30 mins. VLK, KD-VLK, DIA1, FAM69c and denatured controls were administered at concentrations of 0.1 mg/mL PAP was administered at 250 mU (12). APV was administered at 0.2 mg/mL.

Radiant heat sensitivity was determined using the Hargreaves method (50). Mice were placed on a warmed glass floor (29° C.) 20 minutes before each testing and, using a Hargreaves apparatus (IITC Model 390), a focused beam of high-intensity light was aimed at the plantar nonglabrous surface of the hind paws. The intensity of the light was set to 30% of maximum with a cutoff value of 20 seconds. The latency to withdraw either hind paw was measured to the nearest 0.01 seconds. The withdrawal latencies for both paws were averaged for each animal.

Heat tolerance/sensitivity was measured using the Hot Plate assay. A day prior to testing, mice were habituated to the Hot Plate Plexiglass box (IITC) for 10 minutes. For testing, mice were placed individually on the hot plate, heated to either 50° C. or 55° C., and the latency to first sign of paw licking or jumping was recorded. The experiment was iterated 3 times with at least 48 hours between measurements. Stimulus cutoffs of 45 sec and 1 min were used respectively for 50° C. and 55° C.

Cold sensitivity was measured using the cold plantar assay (51). Mice were habituated for 30 min to plexiglass chambers with breathing holes resting atop a ⅛th in thick glass base. The cold probe was made of finely crushed dry ice packed into a modified 3 mL BD syringe. Mice were tested by pressing the cold probe against the glass directly underneath the center of the hindpaw, and latency to withdraw the paw from the cold glass was recorded with a maximum cutoff time of 20 sec. Mice were tested 4 times with at least 15 min between trials.

Sensorimotor evaluation was done using the rotarod test. Mice were acclimated to the Rotarod apparatus (IITC) a day before testing for 5 min at 4 RPM. For testing, mice were placed on the rotating rod and the RPM increased from 4 to 40 at a constant rate across 5 min. Latency to fall, RPM at fall and total distance traveled were all recorded automatically by the apparatus. 3 trials were conducted, with at least 15 minutes between trials.

Hind-paw incision surgeries were performed on transgenic animals as previously described (6, 52). Briefly, a ˜5 mm incision was made on the plantar skin of the left hindpaw under isoflurane anesthesia. The flexor digitorum brevis muscle was briefly lifted (not cut) with forceps, and the incision site was sutured closed.

Sensorimotor evaluation was done using the rotarod test. Mice were acclimated to the Rotarod apparatus (IITC) a day before testing for 5 min at 4 RPM. For testing, mice were placed on the rotating rod, and the RPM increased from 4 to 40 at a constant rate across 5 min. Latency to fall, RPM at fall, and total distance traveled were all recorded automatically by the apparatus. 3 trials were conducted, with at least 15 min between trials.

Antibodies. The following primary antibodies and dilutions were used: mouse monoclonal (IgG1) anti-GluN1 (1:500 (WB), 1:200 (PLA), BioLegend, clone R1JHL, cat #828201, lot #B383911), goat polyclonal anti-EphB2 (1:1200 (PLA), R&D Systems, cat #AF467, lot #CVT0315041), mouse monoclonal (IgG1) anti-EphB2 (1:500 (WB), Thermo Fisher Scientific, clone 1A6C9, cat #37-1700, lot #RD2115698), mouse monoclonal (IgG2b) anti-pTyr (PY99) (1:2000 (WB),Santa Cruz, Cat #sc-7020; lot #L022), rabbit polyclonal anti-FLAG (1:1000 (WB), Sigma, Cat #F7425, lot #078M4886V), mouse monoclonal (IgG2a) anti-PSD-95 (1:2500 (WB), 1:250 (ICC), Neuromab, clone 28/43, cat #75-028, lot #472-2JU/02), mouse monoclonal (IgG1) anti-NeuN (2 mg/mL (IHC), Millipore, clone A60, cat #MAB377), mouse monoclonal (IgG1) anti-Neurofilament 200 (2 mg/mL (IHC), Millipore, clone N52, cat #MAB5266), guinea pig recombinant monoclonal (IgG2k) anti-c-Fos (1:10000 (IHC), Synaptic Systems, cat #226308), mouse monoclonal (IgG1) anti-Synaptophysin-1 (1:5000 (WB), Synaptic Systems, clone 7.2, cat #101 111, lot #1-46), mouse monoclonal (IgG1) anti-Synaptotagmin-1 (1:2000 (WB), Synaptic Systems, Cat #105011, lot #1-47), rabbit polyclonal anti-Synaptotagmin-5/9 (1:2000 (WB), Synaptic Systems, Cat #105053, lot #1-8), rabbit polyclonal anti-RFP (1:2000 (WB), Rockland, Cat #600-401-379, lot #46510), mouse monoclonal (IgG1) anti-GAPDH (1:500 (WB), Millipore, Cat #MAB374, lot #4045089), rabbit polyclonal anti-Tubulin (1:10,000 (WB), Abcam, Cat #ab18251, lot #GR235480-2), rabbit polyclonal anti-Flotilin (1:1000, Novus Bio, Cat #NBP187498, lot #000020065), mouse monoclonal (IgG2b) anti-actin (1:2000 (ICC), Biolegend, clone 2F-1, Cat #643801, lot #B266258), anti-Rabbit VLK (1:2000 (WB), generated by the inventors), anti-rabbit EphB2-pY662 (1:2000 (WB), previously described (9)), guinea pig polyclonal anti-vesicular glutamate transporter 1 (vGlut1; 1:5000 (ICC), Millipore, Cat #AB5905, lot #3987239).

The following secondary antibodies were used: Donkey anti-mouse-HRP (1:10,000 (WB), Jackson ImmunoResearch, cat #715035151 lot #151303), Donkey anti-rabbit-HRP (1:10,000 (WB), Jackson ImmunoResearch, cat #711035152, lot #163128), Goat anti-guinea pig DyLight-488 (1:5000 (PLA), Abcam, cat #ab102374, lot #1041504), Donkey anti-mouse AlexaFluor-647 (1:500 (ICC), Jackson ImmunoResearch, cat #715605150, lot #160478), Goat anti-Mouse IgG1 AlexaFluor-555 (1:500 (IHC), Thermo Fisher Scientific, cat #21127), Goat anti-Mouse IgG1 AlexaFluor-647 (1:500 (IHC), Thermo Fisher Scientific, cat #21240), Goat anti-Guinea pig AlexaFluor-647 (1:500 (IHC), Thermo Fisher Scientific, cat #A21450).

Statistical analysis. GraphPad Prism Software was used to carry out statistical analyses and generate graphs. The respective figure legends indicate the number of subjects used and the statistical analysis, including posthoc comparison tests, used for individual experiments. Mice were randomly allocated to experimental groups when possible. Behavioral testing was carried out by investigators blinded to treatment or genotype, and different experimenters successfully reproduced the results. P values of less than 5% were considered statistically significant, with * representing P<0.05, ** representing P<0.01, *** representing P<0.005, and **** representing P<0.001 unless otherwise indicated. All the models were made using Biorender.

### Example 2—Results

The inventors demonstrated that VLK, among the VLK/PKDCC family of tyrosine kinases, is uniquely capable of inducing the EphB-NMDAR interaction. This was evidenced by co-immunoprecipitation experiments in various neuronal models, including primary cortical neurons and iPSC-derived DRG sensory neurons. Additionally, VLK's role in pain modulation was underscored by the finding that its secretion, regulated by ephrin-B2, modulates synaptic interactions essential for pain perception.

The inventors' research has elucidated that VLK, localized to synaptic vesicles, is released from neurons in a SNARE-dependent fashion. This presynaptic release of VLK induces postsynaptic EphB2-NMDAR interaction, a novel mechanism in the regulation of synaptic protein-protein interactions. This mechanism is further supported by the observation that VLK is secreted from neurons upon ephrin-B2 activation, and its kinase activity is necessary for inducing the EphB2-NMDAR interaction. These findings are validated in human iPSC DRG neurons, in human tissue, and mouse and rat models.

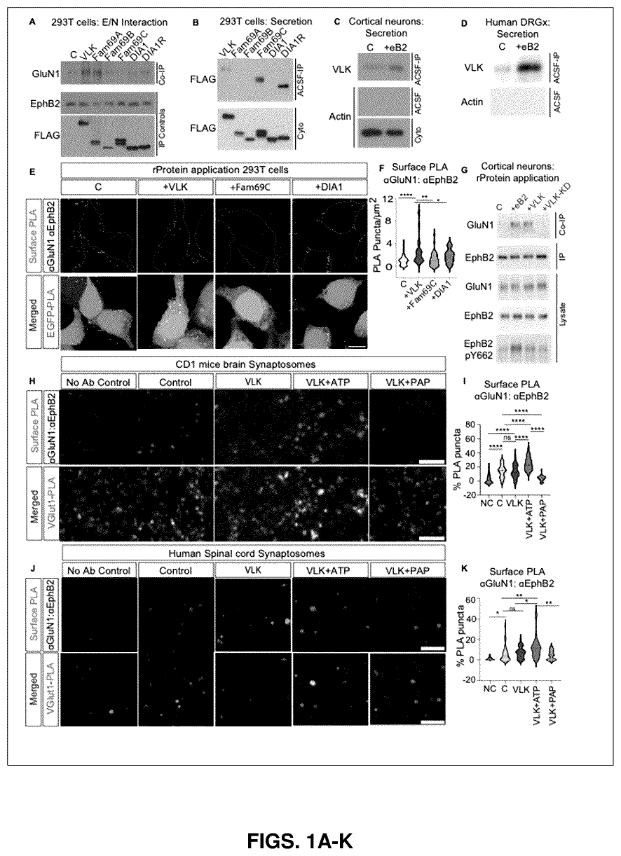

VLK's unique ability to induce the EphB-NMDAR interaction was substantiated through co-immunoprecipitation experiments across various neuronal models. This includes primary cortical neurons, human induced pluripotent stem cell (iPSC)-derived dorsal root ganglion (DRG) sensory neurons (iSNs), and spinal cord synapses, as detailed in FIG. 1A and supplemental figures. These experiments demonstrated that only VLK and Fam69A, among six tested kinases, were able to induce this interaction. These data suggest that other members of this family of kinases may induce phosphorylation.

VLK's role in modulating pain was highlighted through the discovery that its secretion, regulated by ephrin-B2, is essential for pain perception. This was particularly evident in the context of the EphB2-NMDAR interaction at synaptic sites, a critical component in pain signaling pathways.

The application of the inventors' findings extends to human biology. For instance, VLK was found to be secreted by rat cortical neurons and human iSNs upon ephrin-B2 stimulation, implicating its role in human neurological functions (FIG. 1C). Additionally, the use of human spinal cord synaptosomes provided further insights into VLK's role in human neurophysiology.

FIGS. 1A-K show a detailed analysis using HEK293T cells and neuronal models. FIG. 1A shows that VLK and Fam69A, among six kinases, uniquely induced the EphB-NMDAR interaction in HEK293T cells, suggesting their specific role in synaptic modulation. FIG. 1B shows that after overexpression of six kinases in HEK293T cells followed by FLAG immunoprecipitation. only VLK, Fam69C, and DIA1 were secreted, highlighting VLK's unique secretion profile. FIG. 1C shows that in rat cortical neurons and human iSNs, VLK secretion was found to be ephrin-B2 dependent, marking its pivotal role in neuron-specific activities. FIGS. 1E and F show that the addition of recombinant VLK to HEK293T cells induced the EphB2-NMDAR interaction on the cell surface, emphasizing VLK's functionality in synaptic interaction.

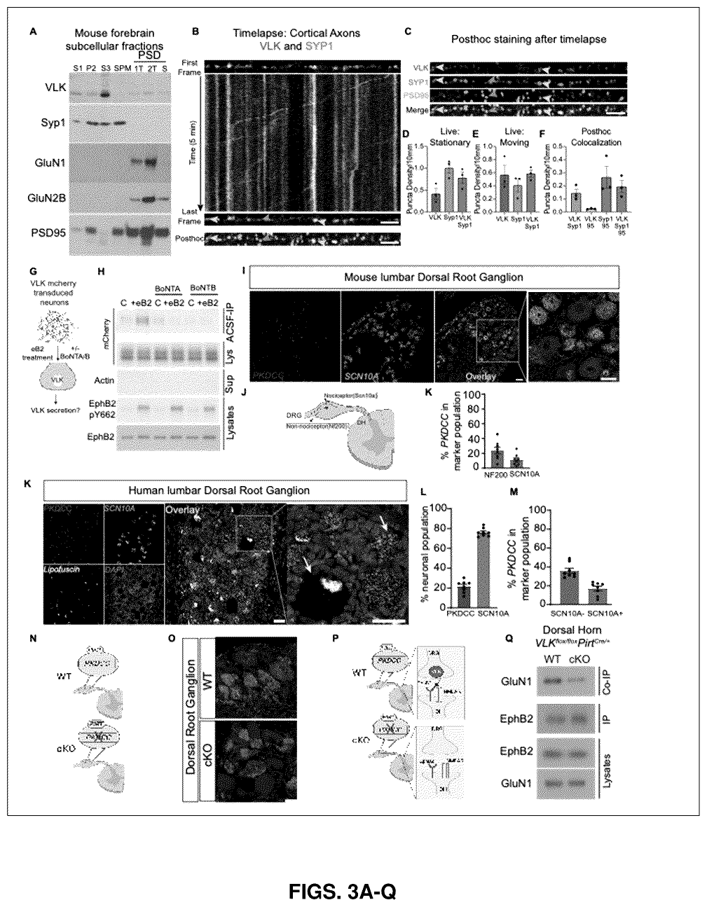

FIGS. 3A-Q examine VLK in synaptosomes and human DRG neurons. FIG. 3A shows VLK co-fractioned with clear-core synaptic vesicles, aligning with its role in synaptic vesicle dynamics. FIG. 3B shows time-lapse imaging of neurons co-expressing VLK-mCherry and synaptic vesicle markers highlighted VLK's dynamic localization within axons. FIG. 3F shows post hoc staining for synaptic markers further confirmed VLK's synaptic localization. FIGS. 3G and H show experiments with BoNT-A and BoNT-B demonstrated the SNARE-dependent mechanism of VLK secretion.

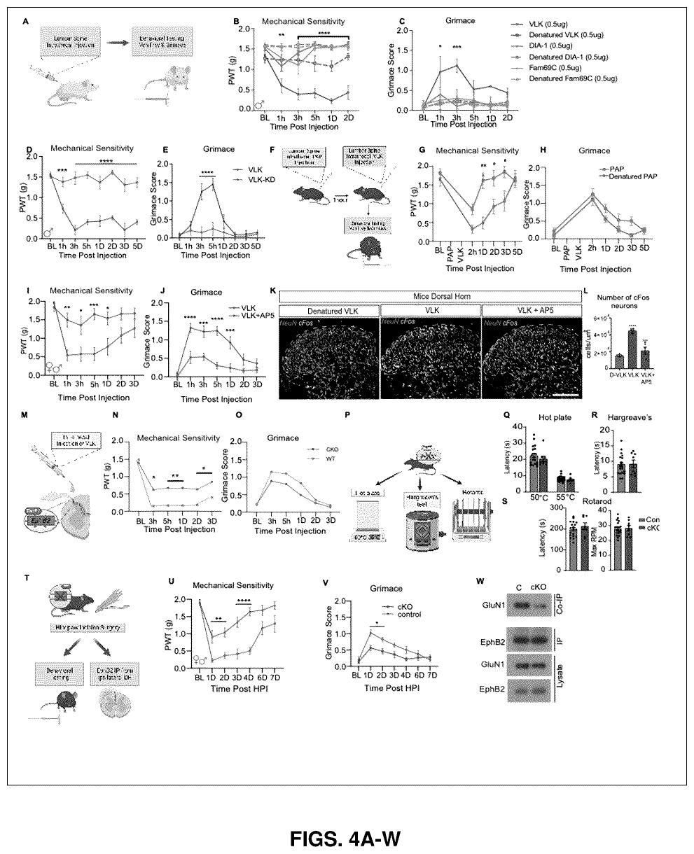

FIGS. 4A-W show data from a model of injury-induced pain and VLK's role in pain management. FIG. 4A shows intrathecal injection model that establishes the direct impact of VLK on spinal cord physiology. FIGS. 4B-C show mechanical sensitivity assessment post-rVLK injection in mice underscored VLK's involvement in pain hypersensitivity. FIGS. 4D and E show the specificity of VLK's kinase activity in pain modulation was confirmed using heat-denatured and kinase-dead VLK variants. FIGS. 4F-H show pre-treatment with PAP before rVLK injection that provided insights into the regulation of VLK-induced pain responses. FIGS. 4I-J show the combination of rVLK with NMDAR antagonists delineated the pathway of VLK-induced pain signaling. FIGS. 4K-J show the analysis of c-fos expression post-VLK injection established the correlation between VLK activity and pain-related neuronal activation. FIGS. 4P-Q show hind paw incision surgery in VLK-KO and WT mice revealed VLK's essential role in post-surgical pain signaling. FIG. 4R shows the assessment of facial grimacing in VLK-KO mice post-surgery provided behavioral evidence of VLK's role in pain modulation.

The inventors research has elucidated that VLK, localized to synaptic vesicles, is released from neurons in a SNARE-dependent fashion. This presynaptic release of VLK induces postsynaptic EphB2-NMDAR interaction, a novel mechanism in the regulation of synaptic protein-protein interactions. This mechanism is further supported by the observation that VLK is secreted from neurons upon ephrin-B2 activation, and its kinase activity is necessary for inducing the EphB2-NMDAR interaction.

FIGS. 3A-Q show VLK's synaptic localization and secretion mechanism. FIG. 3A shows VLK's co-fractionation with clear-core synaptic vesicles in the crude synaptic vesicle fraction suggests its presynaptic localization. This is further confirmed by targeted mass spectrometry analysis. FIG. 3B shows time-lapse imaging showing VLK-mCherry co-localization with synaptic vesicle markers (Syp1-EGFP) in axons indicates VLK's dynamic presence in synaptic vesicle trafficking. FIG. 3F shows post hoc staining for synaptic markers (PSD-95, VLK, Syp1) further supports VLK's synaptic site localization. FIGS. 3G-H shows experiments using BoNT-A and BoNT-B toxins, which cleave SNAP-25 and synaptobrevin respectively, demonstrate VLK's SNARE-dependent secretion mechanism.

VLK in EphB2-NMDAR interaction and phosphorylation is shown in FIGS. 1A-K and FIGS. 2A-C. FIGS. 1E-F show proximity ligation assays on non-permeabilized HEK293T cells indicate that rVLK application induces the EphB2-NMDAR interaction on the cell surface, an effect not found with kinase-dead VLK. FIG. 1I shows treatment of untransfected rat cortical neurons with activated ephrin-B2 and rVLK induces the endogenous EphB2-NMDAR interaction, confirming VLK's role in vivo. FIGS. 2A-C show in vitro cell-free kinase assays show rVLK's unique capacity to phosphorylate the EphB2 ectodomain, crucial for the EphB2-NMDAR interaction.

FIG. 1C and FIGS. 4G-H examiner VLK secretion and activation. FIG. 1C shows that the ephrin-B2 dependent secretion of endogenous VLK in both rat cortical neurons and human iSNs, signifies its regulated release in response to specific neuronal stimuli. FIGS. 4G-H explore the role of extracellular phosphatases like PAP in modulating VLK activity, showing how PAP pre-treatment can block rVLK-induced mechanical hypersensitivity.

FIGS. 4A-R look at VLK's role in pain mechanisms. FIGS. 4-C show intrathecal injection model and subsequent mechanical sensitivity tests in mice illustrate VLK's direct involvement in pain hypersensitivity. FIGS. 4D-E and I-K use heat-denatured and kinase-dead rVLK variants, in combination with NMDAR antagonists, to delineate the dependency of pain-like behavior on VLK's kinase activity and NMDAR interaction. FIGS. 4P-Q and 4R show a hind paw incision model in mice, including observations of mechanical hypersensitivity and facial grimacing, underscores VLK's critical role in injury-induced pain signaling.

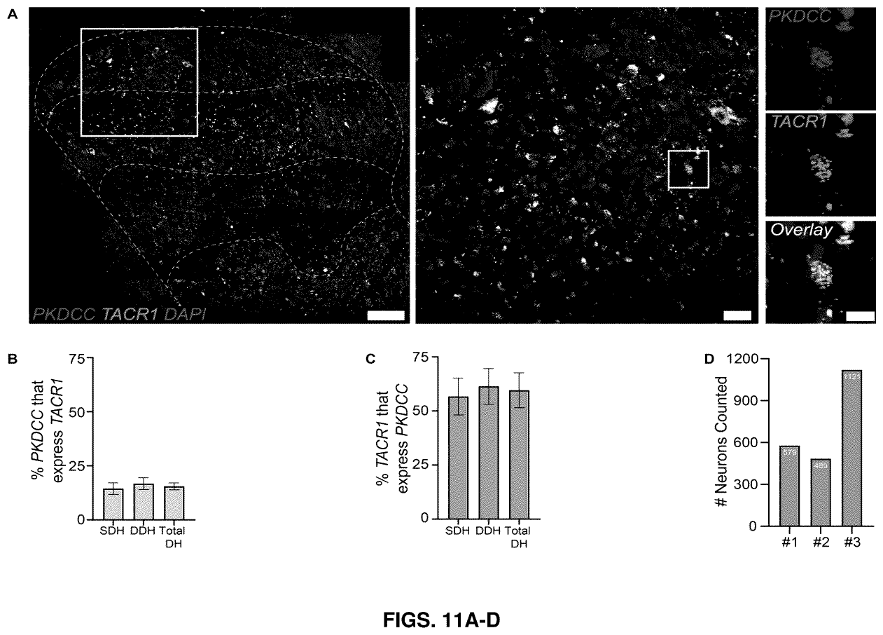

TABLE S1
Donor information for tissues used in
human characterization experiments.

| Donor | | | | Cause of | |
|---|---|---|---|---|---|
| ID | Age | Sex | Ethnicity | Death | Experiment |
| 1 | 51 | M | White | CVA/Stroke | FIGS. 3A-Q |
| 2 | 61 | F | White | Anoxia/Cardiac | FIGS. 3A-Q |
| | | | | Arrest | |
| 3 | 19 | M | White | Anoxia/Drug | FIGS. 3A-Q |
| | | | | Overdose | |
| 4 | 30 | M | Black | Anoxia/Asthma | FIGS. 3A-Q |
| | | | | attack | |
| 5 | 34 | M | White | CVA/Stroke | FIGS. 3A-Q |
| 6 | 34 | F | White | CVA/Stroke | FIGS. 3A-Q |
| 7 | 41 | F | White | Head Trauma/ | FIGS. 3A-Q |
| | | | | Motorcycle | |
| | | | | Accident | |
| 8 | 36 | F | Black | Head Trauma/ | FIGS. 3A-Q |
| | | | | Blunt Injury/MVA | |
| 9 | 47 | M | White | CVA/Stroke | FIGS. 11A-D |
| 10 | 45 | F | White | Anoxia/Cardiac | FIGS. 11A-D |
| | | | | Arrest | |
| 11 | 19 | M | Hispanic | Head Trauma/GSW | FIGS. 11A-D |

TABLE S2
Primers

| Species | Gene | Primer |
|---|---|---|
| Human | PKDCC (VLK) | Fwd 5′-GAAGCGGAACCTCTATAATGCC-3′ (SEQ ID |
| | | NO: 7) |
| Human | PKDCC (VLK) | Rev 5′-GGATACACTGGTACTCGGTACT-3′ (SEQ ID |
| | | NO: 8) |
| Human | DIPK1C (Fam69C) | Fwd 5′-GAGGAGTACGTCTACTTCAGCC-3′ (SEQ ID |
| | | NO: 9) |
| Human | DIPK1C (Fam69C) | Rev 5′-CGGTGGGAAAAGTCACTGTC-3′ (SEQ ID NO: |
| | | 10) |
| Human | DIPK2B (Dia1R) | Fwd 5′-TGGACTGCCTCCCGATTCCT-3′ (SEQ ID NO: |
| | | 11) |
| Human | DIPK2B (Dia1R) | Rev 5′-GGCTGATGCAGAGGCACAGA-3′ (SEQ ID NO: |
| | | 12) |
| Human | GAPDH | Fwd 5′-ATGGGGAAGGTGAAGGTCG-3′ (SEQ ID NO: |
| | | 13) |
| Human | GAPDH | Rev 5′-GGGGTCATTGATGGCAACAATA-3′ (SEQ ID |
| | | NO: 14) |
| Mouse | Pkdcc (VLK) | Fwd 5′-CGGTCACCCTGCTTGACTTC-3′ (SEQ ID NO: |
| | | 15) |
| Mouse | Pkdcc (VLK) | Rev 5′-GGCATTGTAGAGGTTCCGTTTC-3′ (SEQ ID |
| | | NO: 16) |
| Mouse | Dipk1c (Fam69C) | Fwd 5′-GCCCTCAGCTTCCTGGACAT-3′ (SEQ ID NO: |
| | | 17) |
| Mouse | Dipk1c (Fam69C) | Rev 5′-ACGTCAATAGCTACCACCGT-3′ (SEQ ID NO: |
| | | 18) |
| Mouse | Dipk2b (Dia1R) | Fwd 5′-GCCAGCTTCTCTCCCTCCTT-3′ (SEQ ID NO: |
| | | 19) |
| Mouse | Dipk2b (Dia1R) | Rev 5′-AGGTGGGAAGCCAGTGAGTT-3′ (SEQ ID NO: |
| | | 20) |
| Mouse | Hprt | Fwd 5′-TCAGTCAACGGGGGACATAAA-3′ (SEQ ID |
| | | NO: 21) |
| Mouse | Hprt | Rev 5′-GGGGCTGTACTGCTTAACCAG-3′ (SEQ ID |
| | | NO: 22) |
| Mouse | 5′ LoxP Pkdcc | Fwd 5′-CTACAGATAATCTCCTTTAATGGGTGGG-3′ |
| | | (SEQ ID NO: 23) |
| Mouse | 5′ LoxP Pkdcc | Rev 5′-CTTCTACGCAGAGTACTTCAGAAACC-3′ (SEQ |
| | | ID NO: 24) |
| Mouse | 3′ LoxP Pkdcc | Fwd 5′-GACTCCTTGGTCTTACGGAG-3′ (SEQ ID NO: |
| | | 25) |
| Mouse | 3′ LoxP Pkdcc | Rev 5′-CTCCACCAGCTTTAGCCAGC-3′ (SEQ ID NO: |
| | | 26) |

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### V. REFERENCES

The following references, to the extent that they provide exemplary procedural or other details supplementary to those set forth herein, are specifically incorporated herein by reference.
1. V. S. Tagliabracci et al., A Single Kinase Generates the Majority of the Secreted Phosphoproteome. Cell 161, 1619-1632 (2015).
2. V. S. Tagliabracci et al., Secreted kinase phosphorylates extracellular proteins that regulate biomineralization. Science 336, 1150-1153 (2012).
3. Y. Imuta, N. Nishioka, H. Kiyonari, H. Sasaki, Short limbs, cleft palate, and delayed formation of flat proliferative chondrocytes in mice with targeted disruption of a putative protein kinase gene, Pkdcc (AW548124). Dev Dyn 238, 210-222 (2009).
4. H. R. Washburn, N. L. Xia, W. Zhou, Y. T. Mao, M. B. Dalva, Positive surface charge of GluN1 N-terminus mediates the direct interaction with EphB2 and NMDAR mobility. Nat Commun 11, 570 (2020).
5. H. R. Washburn, P. Chander, K. D. Srikanth, M. B. Dalva, Transsynaptic Signaling of Ephs in Synaptic Development, Plasticity, and Disease. Neuroscience 508, 137-152 (2023).
6. K. Hanamura et al., Extracellular phosphorylation of a receptor tyrosine kinase controls synaptic localization of NMDA receptors and regulates pathological pain. PLoS Biol 15, e2002457 (2017).
7. M. J. Nolt et al., EphB controls NMDA receptor function and synaptic targeting in a subunit-specific manner. J Neurosci 31, 5353-5364 (2011).
8. M. S. Kayser, A. C. McClelland, E. G. Hughes, M. B. Dalva, Intracellular and transsynaptic regulation of glutamatergic synaptogenesis by EphB receptors. J Neurosci 26, 12152-12164 (2006).
9. M. B. Dalva et al., EphB receptors interact with NMDA receptors and regulate excitatory synapse formation. Cell 103, 945-956 (2000).
10. C. Minkin, Bone acid phosphatase: tartrate-resistant acid phosphatase as a marker of osteoclast function. Calcif Tissue Int 34, 285-290 (1982).
11. H. Rico, L. F. Villa, Serum tartrate-resistant acid phosphatase (TRAP) as a biochemical marker of bone remodeling. Calcif Tissue Int 52, 149-150 (1993).
12. M. J. Zylka et al., Prostatic acid phosphatase is an ectonucleotidase and suppresses pain by generating adenosine. Neuron 60, 111-122 (2008).
13. P. Guedes-Dias, E. L. F. Holzbaur, Axonal transport: Driving synaptic function. Science 366, (2019).
14. N. Ahimsadasan, V. Reddy, M. Z. Khan Suheb, A. Kumar, in StatPearls. (StatPearls Publishing Copyright @ 2024, StatPearls Publishing LLC., Treasure Island (FL), 2024).
15. S. I. Sheffler-Collins, M. B. Dalva, EphBs: an integral link between synaptic function and synaptopathies. Trends Neurosci 35, 293-304 (2012).
16. A. Y. Kim et al., Pirt, a phosphoinositide-binding protein, functions as a regulatory subunit of TRPV1. Cell 133, 475-485 (2008).
17. A. Latremoliere, C. J. Woolf, Central sensitization: a generator of pain hypersensitivity by central neural plasticity. J Pain 10, 895-926 (2009).
18. S. N. Hassler et al., The cellular basis of protease-activated receptor 2-evoked mechanical and affective pain. JCI Insight 5, (2020).
19. E. V. Brown, A. F. Malik, E. R. Moese, A. F. McElroy, A. C. Lepore, Differential Activation of Pain Circuitry Neuron Populations in a Mouse Model of Spinal Cord Injury-Induced Neuropathic Pain. J Neurosci 42, 3271-3289 (2022).
20. A. Barik et al., A spinoparabrachial circuit defined by Tacr1 expression drives pain. Elife 10, (2021).
21. S. Choi et al., Parallel ascending spinal pathways for affective touch and pain. Nature 587, 258-263 (2020).
22. A. M. Bell et al., Deep sequencing of Phox2a nuclei reveals five classes of anterolateral system neurons. bioRxiv, (2023).
23. O. Gautier et al., Challenges of profiling motor neuron transcriptomes from human spinal cord. Neuron 111, 3739-3741 (2023).
24 J. A. Blum et al., Single-cell transcriptomic analysis of the adult mouse spinal cord reveals molecular diversity of autonomic and skeletal motor neurons. Nat Neurosci 24, 572-583 (2021).
25. T. L. Daigle et al., A Suite of Transgenic Driver and Reporter Mouse Lines with Enhanced Brain-Cell-Type Targeting and Functionality. Cell 174, 465-480.e422 (2018).
26. T. Huang et al., Identifying the pathways required for coping behaviours associated with sustained pain. Nature 565, 86-90 (2019).
27. B. S. Khakh, Molecular physiology of p2× receptors and atp signalling at synapses. Nature Reviews Neuroscience 2, 165-174 (2001).
28. J. P. Dupuis, O. Nicole, L. Groc, NMDA receptor functions in health and disease: Old actor, new dimensions. Neuron 111, 2312-2328 (2023).
29. R. C. Malenka, R. A. Nicoll, NMDA-receptor-dependent synaptic plasticity: multiple forms and mechanisms. Trends Neurosci 16, 521-527 (1993).
30. B. C. Park, M. Reese, V. S. Tagliabracci, Thinking outside of the cell: Secreted protein kinases in bacteria, parasites, and mammals. IUBMB Life 71, 749-759 (2019).
31. R. Ruscheweyh, O. Wilder-Smith, R. Drdla, X.-G. Liu, J. Sandkühler, Long-Term Potentiation in Spinal Nociceptive Pathways as a Novel Target for Pain Therapy. Molecular Pain 7, 1744-8069-1747-1720 (2011).
32. C. N. Sang, NMDA-Receptor Antagonists in Neuropathic Pain: Experimental Methods to Clinical Trials. Journal of Pain and Symptom Management 19, 21-25 (2000).
33. M. Hruska, N. Henderson, S. J. Le Marchand, H. Jafri, M. B. Dalva, Synaptic nanomodules underlie the organization and plasticity of spine synapses. Nature Neuroscience 21, 671-682 (2018).
34. Y. T. Mao et al., Filopodia Conduct Target Selection in Cortical Neurons Using Differences in Signal Kinetics of a Single Kinase. Neuron 98, 767-782.e768 (2018).
35. M. R. Bordoli et al., A secreted tyrosine kinase acts in the extracellular environment. Cell 158, 1033-1044 (2014).
36. P. Chander, M. J. Kennedy, B. Winckler, J. P. Weick, Neuron-Specific Gene 2 (NSG2) Encodes an AMPA Receptor Interacting Protein That Modulates Excitatory Neurotransmission. eneuro 6, ENEURO.0292-0218.2018 (2019).
37. D. Guez-Barber et al., FACS purification of immunolabeled cell types from adult rat brain. J Neurosci Methods 203, 10-18 (2012).
38. M. Hruska, N. T. Henderson, N. L. Xia, S. J. Le Marchand, M. B. Dalva, Anchoring and synaptic stability of PSD-95 is driven by ephrin-B3. Nat Neurosci 18, 1594-1605 (2015).
39. A. J. B. Kreutzberger et al., Reconstitution of calcium-mediated exocytosis of dense-core vesicles. Science Advances 3, e1603208 (2017).
40. J. Rappsilber, M. Mann, Y. Ishihama, Protocol for micro-purification, enrichment, pre-fractionation and storage of peptides for proteomics using StageTips. Nat Protoc 2, 1896-1906 (2007).
41. Y. Xu et al., Cardiolipin remodeling enables protein crowding in the inner mitochondrial membrane. Embo j 40, e108428 (2021).
42. J. Cox et al., Andromeda: a peptide search engine integrated into the MaxQuant environment. J Proteome Res 10, 1794-1805 (2011).
43. S. Tyanova, T. Temu, J. Cox, The MaxQuant computational platform for mass spectrometry-based shotgun proteomics. Nat Protoc 11, 2301-2319 (2016).
44. R. Craig, J. P. Cortens, R. C. Beavis, Open source system for analyzing, validating, and storing protein identification data. J Proteome Res 3, 1234-1242 (2004).
45. S. D. Sherrod et al., Label-free quantitation of protein modifications by pseudo selected reaction monitoring with internal reference peptides. J Proteome Res 11, 3467-3479 (2012).
46. S. Shiers, R. M. Klein, T. J. Price, Quantitative differences in neuronal subpopulations between mouse and human dorsal root ganglia demonstrated with RNAscope in situ hybridization. PAIN 161, 2410-2424 (2020).
47. S. I. Shiers et al., Convergence of peptidergic and non-peptidergic protein markers in the human dorsal root ganglion and spinal dorsal horn. Journal of Comparative Neurology 529, 2771-2788 (2021).
48. S. R. Chaplan, F. W. Bach, J. W. Pogrel, J. M. Chung, T. L. Yaksh, Quantitative assessment of tactile allodynia in the rat paw. J Neurosci Methods 53, 55-63 (1994).
49. D. J. Langford et al., Coding of facial expressions of pain in the laboratory mouse. Nature Methods 7, 447-449 (2010).
50. M. Cheah, J. W. Fawcett, M. R. Andrews, Assessment of Thermal Pain Sensation in Rats and Mice Using the Hargreaves Test. Bio-protocol 7, e2506 (2017).
51. D. S. Brenner, J. P. Golden, R. W. t. Gereau, A novel behavioral assay for measuring cold sensation in mice. PLoS One 7, e39765 (2012).
52. A. M. Cowie, C. L. Stucky, A Mouse Model of Postoperative Pain. Bio-protocol 9, e3140 (2019).

1. A method of targeting Vertebrate Lonesome Kinase (VLK) activity in a subject for the treatment of acute pain or for the treatment of a neurological disorder, comprising delivering to said subject a therapy that blocks VLK kinase activity or activation or that interferes with VLK interaction with EphB receptors,

2. The method of claim 1, wherein the pain is induced by disease, injury and/or surgery, and/or wherein the pain is acute pain, cancer pain, neuropathic pain, injury-induced pain, or NMDAR dependent pain.

3. The method of claim 1, wherein the neurological disorder is linked to the EphB-NMDAR interaction selectively induced by Vertebrate Lonesome Kinase (VLK) and the comprises delivering to said subject a therapy that blocks VLK kinase activity or activation or that interferes with VLK interaction with EphB receptors.

4. The method of claim 1, wherein delivering comprises delivery to thalamus, anterior cingulate cortex, dorsal root ganglion and/or spinal cord of the subject.

5. The method of claim 1, wherein delivering comprises delivery to brain cortex or delivery to the limbic system of the subject.

6. The method of claim 1, wherein the therapy is a drug blocking VLK kinase activity, an antibody or fragment thereof blocking VLK activity, a drug or antibody that interferes with VLK interaction with EphB receptors, or a drug or antibody that interferes with EphB-NMDAR interaction.

7. The method of claim 1, wherein the neurological disorder Alzheimer's, Schizophrenia, NMDAR encephalitis, autism spectrum disorder, or stroke.

8. The method of claim 1, wherein the subject is a mammal, such as a human.

9. (canceled)

10. The method of claim 1, wherein the step of delivering is repeated, such as repeated 1, 2, 3, 4, 5, 6, 7, 8, 9, 10, 15, 20, 25, 50, 75, or 100 times or is given chronically, such as daily or weekly over 1, 2, 3, 4, 5, 6, 7, 8, 9, 10, 11, 12 or 24 months.

11. A method of treating a chronic disease associated with EphrinB upregulation in a subject comprising targeting Vertebrate Lonesome Kinase (VLK) activity in said subject with a drug blocking activation of VLK kinase activity, an antibody or fragment thereof blocking VLK activity, a drug or antibody that interferes with VLK interaction with EphB receptors, or a drug or antibody that interferes with EphB-NMDAR interaction.

12. The method of claim 11, wherein the disease is characterized by EphrinB driving VLK release from dorsal root ganglion peripheral neurons and/or from central nervous system neurons.

13. The method of claim 11, wherein the disease is rheumatoid arthritis, inflammatory bowel disease, stroke and a neurological disorder.

14. (canceled)

15. The method of claim 11, wherein the subject is a mammal, such as a human.

16. A method for treating rheumatoid arthritis, inflammatory bowel disease, or a neurological disorder such as Alzheimer's, Schizophrenia, NMDAR encephalitis, autism spectrum disorder, or stroke in a subject comprising inhibiting JAK/STAT signaling to impair Vertebrate Lonesome Kinase (VLK) activity.

17. The method of claim 16, wherein inhibiting JAK/STAT signaling comprises administration of a drug blocking activating VLK kinase activity, an antibody or fragment thereof blocking VLK activity, a drug or antibody that interferes with VLK interaction with EphB receptors, or a drug or antibody that interferes with EphB-NMDAR interaction.

18. (canceled)

19. A method of treating pain in a subject in need thereof comprising targeted CRISPR gene excision, viral vector mediated gene knockdown, antisense oligonucleotide or RNA interference to knockout or knockdown Vertebrate Lonesome Kinase (VLK) expression in sensory neurons of said subject.

20. (canceled)

21. A method of determining whether a patient will respond to a VLK-targeting therapeutic comprising:
(a) administering an NMDA receptor antagonist to said subject; and
(b) assessing the presence or absence of an acute analgesic response to an NMDA receptor antagonist,
wherein an acute analgesic response indicates that said subject will respond to a VLK-targeting therapeutic.

22. The method of claim 21, further comprising (c) treating said subject with a VLK-targeting therapeutic when an acute analgesic response occurs.

23. (canceled)

24. The method of claim 21, wherein, in the absence of an acute analgesic response, step (a) is repeated at a higher dose of said NMDA receptor antagonist.

25. (canceled)

26. A method of targeting an agent to an EphB2-NMDAR complex in a subject comprising:
(a) providing an agent linked to Vertebrate Lonesome Kinase (VLK) or a fragment thereof that interacts with EphB2 receptors but lacks VLK activity, and
(b) administering said VLK-linked agent to a subject.

27. (canceled)
